(19) United States
(12) Patent Application Publication
(10) Pub. No.: US 2024/0158839 A1
(43) Pub. Date: May 16, 2024

(54) METHODS FOR IN SITU SEQUENCING

(71) Applicant: PRESIDENT AND FELLOWS OF HARVARD COLLEGE, Cambridge, MA (US)

(72) Inventors: Huy Quoc NGUYEN, Cambridge, MA (US); Shyamtanu CHATTORAJ, Cambridge, MA (US); Chao-ting WU, Cambridge, MA (US); George M. CHURCH, Cambridge, MA (US)

(73) Assignee: PRESIDENT AND FELLOWS OF HARVARD COLLEGE, Cambridge, MA (US)

(21) Appl. No.: 18/281,908

(22) PCT Filed: Mar. 14, 2022
(86) PCT No.: PCT/US2022/020138

§ 371 Date: Sep. 13, 2023

Related U.S. Application Data
(60) Provisional application No. 63/161,165, filed on Mar. 15, 2021.

(51) Int. Cl. C12Q 1/6841; C12Q 1/6874

(52) U.S. Cl. CPC C12Q 1/6841; C12Q 1/6874

(57) ABSTRACT

This disclosure is generally directed to methods for obtaining sequence information at nucleotide resolution with spatial information directly from chromosome(s) in situ.

### CROSS-REFERENCE TO RELATED APPLICATIONS

This application claims benefit under 35 U.S.C. § 119(e) of U.S. Provisional Application No. 63/161,165 filed Mar. 15, 2021, the contents of which are incorporated herein by reference in their entirety.

### GOVERNMENT SUPPORT

This invention was made with government support under grant no. R01 HD091797, grant no. R01 GM123289, and grant no. RM1 HG008525 awarded by National Institutes of Health. The government has certain rights in this invention.

### TECHNICAL FIELD

This disclosure is generally directed to methods for obtaining sequence information at nucleotide resolution with spatial information directly from chromosome(s) in situ.

### BACKGROUND

Next generation sequencing (NGS) methods such as massively parallel sequencing or deep sequencing are methods that are robust enough to sequence an entire human genome in as little as one single day. This is in stark contrast to Sanger synthesis methods, which took decades to sequence the human genome.

Current next generation sequencing (NGS) produces reads from cells but cannot provide spatial information and is often acquired from a population of cells. Current in situ sequencing methods provide spatial information at the single cell level but only as an indirect readout of targeted genomic DNA. See, for example, Payne et al. 2021 (DOI: 10.1126/science.aay3446). Acquiring nucleotide resolution combined with spatial information requires a method that sequences directly off of the chromosome in situ. In addition, having single cell resolution can be important to determine the extent to which a tissue is damaged, how many cells in a tissue harbor specific mutations, and whether the localization of the damaged cell(s) is of concern. This information is difficult to obtain from ex situ next generation sequencing methods, even with single cell resolution, as the spatial context is not maintained. Thus, there remains a need in the art for methods, compositions and systems for simultaneously determining sequence and spatial information from chromosomes in situ.

The present disclosure addresses some of the needs in this area.

### SUMMARY

Provided herein are methods and compositions useful for obtaining sequence information, and optionally spatial information, e.g., regarding the sequence, directly from a chromosome in situ at a single nucleotide resolution. Having a direct readout of DNA in situ is beneficial as this can, for example, allow for the phasing of individuals, where a given allele is attributed to either a paternal or maternal chromosome. The methods and compositions provided herein permit one to, for example, predict or determine the phenotypical outcome of one or more mutations based on whether they are present on the same homologous chromosome. Additionally, methods described herein can also serve as a diagnostic for neurodegenerative diseases involving tandem repeats where the number of repeats is indicative of disease status. Without wishing to be bound by theory, the methods described herein can also identify individual cells in a tissue that can harbor detrimental mutations, as the single cell and direct sequencing methods described herein can yield this information. The methods described herein can also be used a) to assess the efficiency of gene editing (e.g., the methods can be used to assess how completely a tissue has been edited), b) to determine the extent to which viruses have invaded a tissue, and/or c) for DNA forensics (e.g., assessment of DNA copy from crime scene samples, suspect samples, etc.).

The methods described herein can also be used to obtain spatial information. For example, the methods described herein can be used to obtain information regarding where on the chromosome and/or where in the cell a sequence is present.

The methods described herein for producing sequence, and optionally spatial, information are collectively referred to as ‘chromoSEQ’ herein.

Accordingly, provided herein in one aspect is a method of determining sequence information on a chromosome in situ, the method comprising: (i) hybridizing a nucleic acid primer to a strand of a chromosome in situ under conditions that permit extension of the hybridized primer by a polymerase; (ii) extending the hybridized primer in presence of a polymerase with a nucleotide to produce an extended hybridized primer, wherein the nucleotide is complementary to a nucleotide, directly downstream from the hybridized primer, on the chromosome strand hybridized to the primer, and wherein the nucleotide is conjugated with a moiety that permits detection of the nucleotide with an agent that specifically binds to the moiety and is capable of producing a detectable signal; and (iii) contacting the moiety with the agent that specifically binds to the moiety and producing a detectable signal, thereby detecting incorporation of the nucleotide onto the hybridized primer.

In one embodiment of this aspect and all other aspects provided herein, the moiety is an antigen or antibody. In another embodiment of this aspect and all other aspects provided herein, the moiety is an antibody.

In another embodiment of this aspect and all other aspects provided herein, the moiety is conjugated with a nucleotide via a cleavable linker.

In another embodiment of this aspect and all other aspects provided herein, the moiety comprises a detectable label. For example, the moiety is conjugated with one or more nanoparticles comprising a fluorophore.

In another embodiment of this aspect and all other aspects provided herein, the agent is conjugated with a detectable label.

In another embodiment of this aspect and all other aspects provided herein, the agent is conjugated with a docking nucleic acid strand.

In another embodiment of this aspect and all other aspects provided herein, the docking nucleic acid strand is conjugated with a detectable label. For example, the docking nucleic acid strand is conjugated to a nanoparticle, e.g., a nanoparticle comprising a fluorophore.

In another embodiment of this aspect and all other aspects provided herein, the step of detecting comprises producing an amplicon from the docking strand nucleic acid strand and detecting the amplicon.

In another embodiment of this aspect and all other aspects provided herein, the step of detecting comprises a Signal amplification by Exchange Reaction (SABER) amplification from the docking strand nucleic acid strand and detecting the SABER amplified signal. SABER amplification is described, for example, in Kishi et al. (DOI: 10.1038/s41592-019-0404-0), content of which is incorporated herein by reference in its entirety.

In another embodiment of this aspect and all other aspects provided herein, the detecting step comprises hybridizing a reporter nucleic acid strand with the docking nucleic acid strand, wherein the reporter nucleic acid strand comprises a detectable label.

In another embodiment of this aspect and all other aspects provided herein, the agent is an antibody or nanobody.

In another embodiment of this aspect and all other aspects provided herein, the method further comprises a step of extinguishing the detectable signal.

In another embodiment of this aspect and all other aspects provided herein, the method further comprises: (i) extending the extended hybridized primer in presence of a polymerase with a second nucleotide, wherein the second nucleotide is complementary to a nucleotide directly downstream from the extended hybridized primer on the chromosome strand to which the primer is hybridized, and wherein the nucleotide is conjugated with a second moiety that permits detection of the nucleotide with a second agent that specifically binds to the second moiety and is capable of producing a detectable signal; and (ii) contacting the second moiety with the second agent that specifically binds to the moiety and producing a detectable signal, thereby detecting incorporation of the second nucleotide on to the extended hybridized primer.

In another embodiment of this aspect and all other aspects provided herein, the primer comprises a detectable label.

In another embodiment of this aspect and all other aspects provided herein, the primer comprises a barcode sequence.

In another embodiment of this aspect and all other aspects provided herein, the method further comprises detecting hybridization of the primer on the chromosome.

In another embodiment of this aspect and all other aspects provided herein, the primer hybridizes to a repetitive element.

In another embodiment of this aspect and all other aspects provided herein, the repetitive element is selected from the group consisting of a Long Interspersed Nuclear Element (LINE), Short Interspersed Nuclear Elements (SINE), SVA element, Alu element, centromeric repeat, and a telomeric repeat.

In another embodiment of this aspect and all other aspects provided herein, the method further comprises, prior to hybridizing the nucleic acid primer, generating a single-stranded region on the chromosome in situ that permits hybridizing with the primer. The single-stranded region on the chromosome can be generated using natural or unnatural methods.

Naturally-generated single stranded regions include, for example, DNA replication forks, Okazaki fragments, DNA transcription bubbles, telomere ends, nicks caused by enzymes (such as Topoisomerase 2) and DNA damage. Exemplary unnatural or non-natural methods for generating single-stranded regions include, but are not limited to, DNA damage (e.g., UV, chemical, stress, etc.), exonucleases, and CO-FISH/RASER-FISH (e.g., nucleotide analogs such as BrdU, etc.). See, for example, Brown et al. 2018 (DOI: 10.1038/s41467-018-06248-4) and Miron et al. (DOI: 10.1126/sciadv.aba8811), contents of both of which are incorporated herein by reference in their entireties.

In another embodiment of this aspect and all other aspects provided herein, the chromosome is in a cell.

In another embodiment of this aspect and all other aspects provided herein, the cell is in a tissue or section thereof.

In another embodiment of this aspect and all other aspects provided herein, the cell, tissue or section thereof is in a matrix.

Another aspect provided herein relates to a method of determining sequence information on a chromosome in situ, the method comprising: (i) providing a chromosome in situ that has a nicked strand, the nick leaving an extendable terminus; (ii) extending the extendable terminus of the nicked strand of the chromosome in the presence of a polymerase with a nucleotide, wherein the nucleotide is complementary to a nucleotide, directly downstream from the extendable terminus, on a chromosome strand complementary to the nicked strand, and wherein the nucleotide is conjugated with a moiety that permits detection of the nucleotide with an agent that specifically binds to the moiety and is capable of producing a detectable signal; and (iii) contacting the moiety with the agent that specifically binds to the moiety and producing a detectable signal, thereby detecting incorporation of the nucleotide onto the nicked strand.

In one embodiment of this aspect and all other aspects provided herein, further comprising creating a nick on a strand of the chromosome prior to extending with the polymerase and the nucleotide.

In another embodiment of this aspect and all other aspects provided herein, further comprising, prior to extending with the polymerase and the nucleotide, contacting the nicked strand with an exonuclease.

In another embodiment of this aspect and all other aspects provided herein, the polymerase is a strand-displacing polymerase.

In another embodiment of this aspect and all other aspects provided herein, the moiety is an antigen or antibody.

In another embodiment of this aspect and all other aspects provided herein, the moiety is an antibody.

In another embodiment of this aspect and all other aspects provided herein, the moiety is conjugated with the nucleotide via a cleavable linker.

In another embodiment of this aspect and all other aspects provided herein, the agent is conjugated with a detectable label.

In another embodiment of this aspect and all other aspects provided herein, the agent is conjugated with a docking nucleic acid strand.

In another embodiment of this aspect and all other aspects provided herein, said detecting comprises a step of producing an amplicon from the docking strand nucleic acid strand and detecting the amplicon.

In another embodiment of this aspect and all other aspects provided herein, the detecting step comprises hybridizing a reporter nucleic acid strand with the docking nucleic acid strand, wherein the reporter nucleic acid strand comprises a detectable label.

In another embodiment of this aspect and all other aspects provided herein, the agent is an antibody or nanobody.

In another embodiment of this aspect and all other aspects provided herein, the method further comprises a step of extinguishing the detectable signal.

In another embodiment of this aspect and all other aspects provided herein, the method further comprises: (i) further extending the nicked strand in presence of a polymerase with a second nucleotide, wherein the second nucleotide is complementary to a nucleotide, directly downstream from the extended nicked strand, on the chromosome strand complementary to the nicked strand, and wherein the second nucleotide is conjugated with a second moiety that permits detection of the nucleotide with a second agent that specifically binds to the moiety and is capable of producing a detectable signal; and (ii) contacting the second moiety with the second agent that specifically binds to the second moiety and producing a detectable signal, thereby detecting incorporation of the second nucleotide on to the nicked strand.

In some embodiments of any one of the aspects, the incorporation of the nucleotide is detected using the method described in Drmanac et al. bioRxiv preprint doi: https:/doi.org/10.1101/2020.02.19.953307 or US Patent Publication No. US20180223358, content of both is incorporated herein by reference in its entirety.

In another embodiment of this aspect and all other aspects provided herein, the chromosome is in a cell.

In another embodiment of this aspect and all other aspects provided herein, the cell is in a tissue or section thereof.

In another embodiment of this aspect and all other aspects provided herein, the cell, tissue or section thereof is in a matrix.

Another aspect provided herein relates to a method of determining sequence information on a chromosome in situ, the method comprising: (i) hybridizing a molecular inversion probe (MIP) to a strand of a chromosome in situ under conditions that permit extension of the MIP, wherein a first end of the MIP hybridizes to a first region of the chromosome strand and the second end of the MIP hybridizes to a second region of the same chromosome strand and wherein the first and the second regions are separated by at least one nucleotide; (ii) extending one end of the hybridized MIP by at least one nucleotide in presence of a polymerase with a nucleotide, wherein the nucleotide is complementary to a nucleotide, directly downstream from the first or second end of the hybridized MIP, on the chromosome strand hybridized to the MIP; (iii) ligating together the two ends of the hybridized, extended MIP; (iv) amplifying the ligated MIP to generate a template strand; and (v) sequencing the template strand.

In one embodiment of this aspect and all other aspects provided herein, amplifying the MIP comprises rolling circle amplification.

In another embodiment of this aspect and all other aspects provided herein, the MIP comprises a barcode sequence.

In another embodiment of this aspect and all other aspects provided herein, the MIP comprises a priming sequence.

In another embodiment of this aspect and all other aspects provided herein, the step of sequencing the template strand is by a fluorescence-based sequencing method.

In another embodiment of this aspect and all other aspects provided herein, said sequencing of the template strand is sequencing by ligation.

In another embodiment of this aspect and all other aspects provided herein, said sequencing of the template strand is sequencing by hybridization.

In another embodiment of this aspect and all other aspects provided herein, said sequencing of the template strand is sequencing by synthesis.

In another embodiment of this aspect and all other aspects provided herein, the MIP hybridizes to a repetitive element.

In another embodiment of this aspect and all other aspects provided herein, the repetitive element is selected from the group consisting of a Long Interspersed Nuclear Elements (LINE), Short Interspersed Nuclear Elements (SINE), SVA element, Alu element, centromeric repeat, and a telomeric repeat.

In another embodiment of this aspect and all other aspects provided herein, the method further comprises, prior to hybridizing the MIP, generating a single-stranded region on the chromosome for hybridizing with the MIP.

In another embodiment of this aspect and all other aspects provided herein, the chromosome is in a cell.

In another embodiment of this aspect and all other aspects provided herein, the cell is in a tissue or section thereof.

In another embodiment of this aspect and all other aspects provided herein, the cell, tissue or section thereof is in a matrix.

Also described herein in another aspect is a method of determining sequence information on a chromosome in situ, the method comprising: (i) hybridizing a nucleic acid probe to a strand of a chromosome in situ, wherein the nucleic acid probe comprises a barcode sequence, a docking sequence, and a sequence complementary to a nucleotide sequence of the chromosome strand; (ii) hybridizing a molecular inversion probe (MIP) to the docking sequence of the probe under conditions that permit extension of the MIP, wherein a first end of the MIP hybridizes to a first region of the probe and the second end of the MIP hybridizes to a second region of the probe and wherein the first and the second regions are separated by at least one nucleotide; (iii) extending one end of the hybridized MIP by at least one nucleotide in presence of a polymerase with a nucleotide, wherein the nucleotide is complementary to a nucleotide, directly downstream from the first or second end of the hybridized MIP, on the probe; (iv) ligating together two ends of the hybridized, extended MIP; (v) amplifying the ligated MIP to generate a template strand; and (vi) sequencing the template strand.

As described above, the method comprises both ligation and synthesis.

In one embodiment of this aspect and all other aspects provided herein, said amplifying of the MIP comprises rolling circle amplification.

In another embodiment of this aspect and all other aspects provided herein, said sequencing of the template strand is by a fluorescence-based sequencing method.

In another embodiment of this aspect and all other aspects provided herein, wherein said sequencing of the template strand is sequencing by ligation.

In another embodiment of this aspect and all other aspects provided herein, said sequencing of the template strand is sequencing by hybridization.

In another embodiment of this aspect and all other aspects provided herein, said sequencing of the template strand is sequencing by synthesis.

In another embodiment of this aspect and all other aspects provided herein, the MIP hybridizes to a repetitive element.

In another embodiment of this aspect and all other aspects provided herein, the repetitive element is selected from the group consisting of a Long Interspersed Nuclear Elements (LINE), Short Interspersed Nuclear Elements (SINE), SVA element, Alu element, centromeric repeat, trinucleotide repeat, and a telomeric repeat.

In another embodiment of this aspect and all other aspects provided herein, the method further comprises, prior to hybridizing the MIP, generating a single-stranded region on the chromosome for hybridizing with the MIP.

In another embodiment of this aspect and all other aspects provided herein, the chromosome is in a cell.

In another embodiment of this aspect and all other aspects provided herein, the cell is in a tissue or section thereof.

In another embodiment of this aspect and all other aspects provided herein, the cell, tissue or section thereof is in a matrix.

It is noted that the methods described herein are amenable to any method of DNA synthesis. In other words, any method of DNA synthesis and/or sequencing could be used with the methods described herein, which permit detection at single nucleotide resolution. For example, in addition to polymerase-based extension, the methods described herein can be used with chemical extension or ligation of DNA.

Also provided herein is a method for counting the short tandem repeats (STRs) in a target nucleic acid. The method comprises: (i) hybridizing a first oligonucleotide directly adjacent to a STR region in a target nucleic acid; (ii) ligating a second oligonucleotide to the first oligonucleotide that is hybridized with the target, wherein the second oligonucleotide comprises a nucleotide sequence complementary to one STR unit and wherein the second oligonucleotide comprises a detectable label; (iii) detecting the detectable label; (iv) cleaving the ligated second oligonucleotide, e.g., with a nicking enzyme to release the detectable label from the ligated oligonucleotide; and (v) repeating steps (ii)-(iv), until a detectable label is not detected in step (iv).

While the methods described herein comprise extension of a nucleic acid strand (e.g., primer or a nick-strand of a chromosome) using a polymerase, any method for template based nucleic acid extension can be used. In other words, any DNA sequencing methods can be used with the methods of the invention. For example, enzymes other than a polymerase can be used for the incorporation of a nucleotide. Non-limiting examples include ligases and transferases e.g., Tdt (www.neb.com/products/m0315-terminal-transferase#Product %20Information), can be used for the extension step. Non-enzymatic methods can also be used. For example, non-enzymatic methods can be through hybridization, amines [NHS ester chemistry], CLICK chemistry, etc.

In some embodiments of any of the aspects described herein, a chromosome is cut and ligated onto itself to form a circle. The circular chromosome is then rolling circle amplified, e.g., with Phi29. The target is PCR amplified in situ to make local copies that are used for chromoSEQ, amplifying signal.

It is noted that the methods described herein can also be used with unconventional (i.e., nucleobases other than A, C, T, and G) nucleobases. In addition, the methods described herein can also be used for in situ sequencing of synthetic (or unknown) genomes that use unconventional bases. Also, the methods can be used to sequence centromeric regions or other regions that comprise non-G, A, T or C nucleobases. See, for example, Shu et. al. 2018 (DOI: 10.1038/s41589-018-0065-9) reporting the presence of uracil in centromeres.

The methods described herein include extending a nucleic acid strand (e.g., a primer or nicked strand) by a nucleotide prior to detecting the incorporation. It is noted that more than one nucleotide can be incorporated prior to detecting the incorporation. For example, A, C, and/or T can to be incorporated, to get past a region, followed by addition of G to sequence a nucleotide of interest. Without wishing to be bound by a theory, this can reduce the number of sequencing rounds that may be required to identify a sequence of interest.

The methods described herein comprise detecting incorporation of a nucleotide onto a nucleic acid strand (e.g., a primer or nicked strand). Where e the methods described herein comprise contacting a moiety, comprising a detectable label, with the incorporated nucleotide, any method known and/or available for detection can be used. For example, a detectable signal can be emitted or generated by an enzyme used for the extension step, e.g., use of polymerases in pyrosequencing (See, for example, Nyren et al 1985 (DOI:/10.1016/0003-2697(85)90211-8)) or by following a fluorescent enzyme (DNA polymerase, DNA ligase, TdT, etc.).

In some embodiments of any of one of the aspects described herein, the primer can comprise a moiety/label for isolating or purifying the primer (e.g., primer comprising the incorporated nucleotide) for ex situ analysis, such as ex situ next generation sequencing (NGS), mass spectrometry, antibody detection for chromatin mark profiling, etc. For example, the primer can comprise a moiety/label that is capable of being pulled down, e.g. biotin and pull down with streptavidin.

In some embodiments of any one of the aspects, the primer comprises a detectable label. In some embodiments of any one of the aspects, the primer comprises a quencher moiety, e.g., a chemical quencher, such as a blackhole quencher (available on the world wide web at biosearchtech.com/support/education/fluorophores-and-quenchers/black-hole-quencher-dyes), Iowa black (idtdna.com/site/Catalog/Modifications/Category/4, etc.)

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1C. FIG. 1A is a schematic representation of exemplary embodiments related to in situ sequencing directly from a chromosome. Oligonucleotide primers (blue) are hybridized to genomic DNA after denaturation (1-2). Such primers can be designed to any region including repetitive regions such as LINEs, SINEs, centromeric, or telomeric regions. Primers directed to repetitive regions are useful since a common primer targeting consensus sequence can permit extension into regions of the DNA having unique sequences. Thus, a single sequence-specific primer permits sequencing of multiple unique regions of the genomic DNA. In certain embodiments, one or more primers can contain a barcode to permit site-specific mapping. Such primers can also be labeled directly with, for example, fluorophores, matrix attachment moieties for hydrogel embedding, elements for detection by mass spectrometry, or gold nanoparticles for signal amplification. Primers are extended with in situ sequencing (ligation, synthesis, or hybridization) using the genome as a template. The embodiment depicted in this schematic is “sequencing by synthesis” (SBS). After sequencing, a signal can be detected with conventional diffraction limited microscopy or super-resolution microscopy (3). Next, the signal is removed, for example by photobleaching, enzymatic cleavage, light induced cleavage, or chemical cleavage, among others (4). Extension/sequencing continues (5). After sequencing is complete, sequencing reads can be mapped back to a particular site. FIG. 1B shows in situ sequencing directly off chromosomes according to an embodiment as shown in FIG. 1A. Oligonucleotides (oligos) were hybridized to centromeric regions on multiple chromosomes and contained non-genome hybridizing regions that were visualized with a complementary oligonucleotide conjugated to a fluorophore (Alexa 488; green). Centromere-targeting oligos were extended with DNA Polymerase and Alexa-647 labeled dCTP using the chromosome as a template. Here, dCTP does not contain a cleavable bond and signal is extinguished with photobleaching. Labeled nucleotides can be added individually or in combination with unlabeled nucleotides for sequence mapping. FIG. 1C shows in situ sequencing directly off chromosomes according to another embodiment of FIG. 1A using Illumina NextSeq™ chemistry that permits extension of one base from primer extension/sequencing. This technology uses nucleotides having terminators that will allow only a single base of extension. These nucleotides are cleavable and remove signal as well as allow for the next base of extension/sequencing.

FIG. 2 is a schematic representation of another exemplary embodiment where the biological sample is treated with DNA nicking enzymes to form single stranded breaks across the genome (1). Exonuclease treatment exposes larger regions of ssDNA through 3′ to 5′ or 5′ to 3′ resection and the resulting DNA will serve as a template for in situ sequencing (2). Digested and resected regions are extended with in situ sequencing by synthesis (3). After sequencing, signal can be detected with conventional diffraction limited microscopy as well as super-resolution microscopy (3). Next, signal is removed as desired (4). Extension/sequencing continues (5). After sequencing is complete, sequencing reads can be mapped back.

FIGS. 3A-3C. FIG. 3A is a schematic representation of another exemplary embodiment using oligonucleotide molecular inversion probes (MIPs/padlock probes), which are hybridized to the genomic DNA after denaturation (1). MIPs can contain gaps of varying nucleotides to mediate genomic sequence detection in subsequent steps. MIPs can also contain barcodes to aid in location determination as well as to mediate the use of universal sequences for priming. Gaps are polymerized across with dNTPs and DNA polymerase, using genomic sequence as a template (2). After polymerizing across gaps, the MIP is circularized by ligation. Circularized MIPs can also be amplified through rolling circle amplification, producing a rolony, to enhance downstream signal (3). Circularized MIP or resulting rolony can be hybridized with an oligonucleotide primer and used for in situ sequencing by synthesis to localize the MIP and identify nucleotides of the gap (4). After sequencing, signal can be detected with conventional diffraction limited as well as super-resolution microscopy (4). Next, signal is removed as desired (5). Extension/sequencing continues (6). After sequencing is complete, sequencing reads can be mapped back. FIG. 3B shows hybridization and detection of 10 MIPs hybridized to chromosome in situ according to an embodiment of FIG. 3A. MIPs were detected using a fluorophore conjugated oligonucleotide hybridizing to a barcode common to the backbone of all 10 MIPs. Absence of signal in Cy3 channel shown to confirm MIP detection signal. 10 μm scale bar. FIG. 3C shows results of another exemplary embodiment of FIG. 3A comprising MIP hybridization to a non-genome hybridizing region of oligonucleotide probe (Oligopaint™) (1), single nucleotide gap polymerization (2), MIP circularization (3), MIP amplification by Rolling Circle Amplification (RCA) (3), and two cycles of in situ sequencing by ligation (4,5) to sequence across the gap.

FIG. 4 is a schematic representation of an embodiment of a novel sequencing-by-synthesis based method using a three-part system. This exemplary method can be used for in situ as well as ex situ sequencing. DNA to be sequenced (e.g., chromosomes in a nucleus, barcodes on oligonucleotides (targeting genomic DNA, RNA, proteins), DNA on sequencing arrays, etc.) is hybridized with an oligonucleotide primer (blue) (1,2). The primer can optionally contain a barcode to aid in mapping or can be labeled directly with fluorophores, matrix attachment moieties for hydrogel embedding, elements for detection by mass spectrometry, or gold nanoparticles for signal amplification. Sequencing by synthesis (SBS) is performed with antibody conjugated reversible terminator nucleotides to extend primers. Each nucleotide (nt) is conjugated to a specific primary antibody (1Ab) (3). Specific secondary antibodies (2Ab) conjugated to oligonucleotides (20) react to specific antibodies on nucleotides (4). Secondary oligonucleotide sequences can also function as “docking strands” as used in DNA-PAINT™ methods for super-resolution imaging. Labeled oligonucleotides hybridize to and detect oligos on secondary antibodies (5). The labeled oligonucleotides can be imager strands for DNA-PAINT™, labeled with multiple fluorophores to enhance signal, labeled with elements for detection by mass spectrometry, or recruit additional oligonucleotides for signal amplification (HCR, SABER, RCA). Signal can be detected with conventional diffraction limited microscopy as well as super-resolution microscopy (5). Next, signal is removed and a hydroxyl group is exposed for the next based to be sequenced (6). The rest of the sequence is read in the same manner (7).

FIGS. 5A-5C show phasing (FIG. 5A) by way of Homolog specific Oligopaints™ (HOPs), which are exemplary Fluorescent in situ hybridization (FISH) probes. (FIG. 5B) and/or in situ sequencing (FIG. 5C).

FIGS. 6A-6C show exemplary embodiments as described herein using (i) labeling with dye and a NHS modified gold nanoparticle (FIG. 6A), (ii) labeling with DBCO NHS modifier (FIG. 6B), and (iii) click-based conjugation (FIG. 6C). In some embodiments, the primary amine groups present in antibodies are labeled with fluorophore containing, NHS-modified gold nanoparticles. This results in an antibody conjugated with multiple fluorophores. The resultant antibody conjugate can be used to detect single nucleotide incorporation. The presence of multiple fluorophores helps in signal amplification and enhanced detection. In some other embodiments, the primary amine groups present in base-specific antibodies are modified using DBCO-NHS modifier. The NHS modified gold nanoparticles are conjugated with docking nucleic acid strands that contain NH₂ group at the 3′ end and an azide group at the 5′ end, and an internal fluorophore. This results in nanoconjugates containing multiple fluorophores. The DBCO modified antibody is then conjugated to the azide groups present in the nanoconjugate using a click reaction. This results in the antibody containing a high number of fluorophores. This antibody conjugate can be used to detect single nucleotide incorporation. The presence of multiple fluorophores helps in signal amplification and enhanced detection.

FIGS. 7A-7C show ChromoSEQ: ExpoSEQ 3 basepairs of lacO repeat in U2OS cells.

FIG. 7A Human U2OS cells containing an integrated construct with lacO and tetO sequence repeats were subjected to RASER-FISH (Brown, J. M. et al. Nat Commun 9, 3849 (2018)) protocol. RASER-FISH: Cells were grown on coverslips and allowed to adhere for 24 hours. Cell media was replaced with media containing bromo-deoxyuridine (BrdU) and bromo-deoxycytosine (BrdC) for 24-28 hours. Newly replicated DNA strands incorporate BrdU/BrdC during this time. Cells were then fixed with 4% formaldehyde in 1×PBS followed by irradiation under UV light (254 nm), causing DNA breaks on BrdU/BrdC incorporated strands. Exonuclease III was used to excise damaged/nicked DNA, resulting in single stranded genomic DNA. FIG. 7B An oligonucleotide primer containing 30 nt of homology to 30/36 nt of lacO sequence was added to cells and hybridized overnight. The primer contains an overhang and a non-genome hybridizing sequence that can recruit fluorophore labeled oligos allowing for detection. Primer was designed to hybridize to 30/36 nt of lacO repeat such that single base extension off the primer results in the addition of 256 of the same nucleotide. FIG. 7C chromoSEQ was then performed to sequence the first base (“Cycle 1”, expected to be Cytosine; (C)). Phusion DNA Polymerase was added with dCTP labeled with Alexa 647 and imaged, the panel shows 2 nuclei (Cycle 1 dCTP-647). A fluorophore labeled oligo (green) was then hybridized to flanking sequence to confirm location of lacO loci (Cycle 1 LacO). 647 nm signal was then bleached to extinguish signal before proceeding to sequencing next base (Cycle 1 Bleach). Next base (second) predicted to be added is Adenine. Phusion was added with unlabeled dATP and dUTP labeled with Alexa 647 and imaged, showing weak/non-existent signal in the same nuclei, as expected due to lack of fluorophore on Adenine. Note that lack of signal indicates that uridine was not added, as expected. Sample was then bleached. Next base (third) predicted to be added is Cytosine and signal appears after addition of Phusion and dCTP labeled with Alexa 647. Next base (fourth) predicted to be added is Adenine and signal is weak/non-existent after addition of Phusion and dUTP labeled with Alexa 488. Last panel shows signal from fluorophore labeled oligo used to detect the primer, demonstrating that primer is still present.

FIGS. 8A-8D ChromoSEQ; expoSEQ+primeSEQ preliminary data. U2OS cells containing LacO array (256 repeats) were subjected to RASER-FISH to yield single stranded genomic DNA to be used for direct sequencing off chromosomes. FIG. 8A Multiple centromeric repeats (Multi-cent; magenta), 2.4 Mb pericentromeric region of Chr19 (yellow), and 36/36 nt of LacO repeat (green) were hybridized by oligos and visualized with labeled oligos targeting overhangs (streets). Image of 1st round of chromoSEQ using PfU High Fidelity DNA Polymerase with dCTP-Alexa647. Most LacO signal do not colocalize with magenta, indicating lack of sequencing from these targets. This was expected as the LacO 36nt oligo covers the entire LacO sequence. FIG. 8B Similar experiment to FIG. 8A except using a shorter LacO oligo (30nt) replaces the 36nt. The next base added should be Cytosine. Here, LacO and magenta signal colocalize (white), indicating sequencing from these targets and addition of dCTP. FIG. 8C Similar experiment to FIG. 8B except Multi-cent oligos were not added and labeled oligos were not added until after sequencing was performed. FIG. 8D Similar experiment to FIG. 8C except Multi-cent oligos replaced Chr19qPC probes.

FIG. 9 is a schematic showing an exemplary method as described herein. Method uses sequencing-by-synthesis off Oligopaint streets (OligoFISSEQ synthesis based interrogation of targets).

FIGS. 10A-10C shows chromoSEQ primeSEQ 1 basepair at Ghimalia repeat in human U20S (A) and PGP1f (C) cells. FIG. 10A An oligonucleotide primer containing 38 nt homology to the 80 nt reported Ghimalia repeat sequence was added to cells and hybridized overnight. The primer contains an overhang with a non-genome hybridizing sequence that can recruit fluorophore labeled oligos (grey bar with circle) allowing for detection. Primer was designed to hybridize to 38/80 nt of Ghimalia repeat such that single base extension off the primer results in the addition of ˜343 of the same nucleotide. In this case, a “C”. FIG. 10B chromoSEQ was performed in Human U20s cells after standard FISH protocols was used to hybridize oligonucleotide in FIG. 10A. One base was sequenced using Illumina NextSeq chemistry, yielding addition of the expected “C” base in magenta. Colocalization of signal confirms sequencing from the Ghimalia primer oligonucleotide. FIG. 10C chromoSEQ was performed in PGP1f cells after standard FISH protocols were used to hybridize oligonucleotide in FIG. 10A. One base was sequenced using Illumina NextSeq chemistry, yielding addition of the expected “C” base in magenta. Colocalization of signal confirms sequencing from the Ghimalia primer oligonucleotide.

FIGS. 11A-11C Schematic diagrams depicting STR ligation for diagnosis of Huntington's disease (FIG. 11A), 17OligoFISSEQ rounds to quantify and localize CAG repeats (FIG. 11B), detection of signal after round 18 indicates Huntington's disease. This method can be used in multiplex with other repeats by making different STR ‘Just Enough Barcodes (JEBs).’

FIG. 12 A schematic diagram indicating how ex situ use of STR JEBs can complement NGS approaches.

FIG. 13 A schematic diagram demonstrating in situ ligation of specific oligos.

FIG. 14 A schematic diagram depicting 1 round of ligation using genome as template reading gagcg at lacO region. The left panel shows colocalized JEB a647 and 2° a488 signal, indicating ligation at lacO sequencing primer site. The absence of the signal for JEB a647 after cleavage indicates JEB a647 ligation. The right panel shows the absence of a JEB a647 signal in no enzyme control indicates that ligation is required for JEB a647 addition. The presence of 2° a488 indicates that the lacO sequencing primer is hybridized. (Cells: U20S-LacO, Method: RASER FISH, Chem: T3/Quick JEBs, Scope: Nikon).

FIG. 15 shows strategies for signal detection and amplification. Exemplary methods for detecting single ligations can include: JEB overhang for amplification (e.g., RCA, SABER, HCR, CLAMP), or single-molecule localization microscopy (SMLM) (e.g., STORM, DNA PAINT).

### DETAILED DESCRIPTION

The methods and compositions provided herein are based, in part, on the discovery of a method for simultaneously determining the sequence of a given target nucleic acid (e.g., an in situ target nucleic acid) and also determining the spatial information relating to that sequence. The methods and compositions provided herein permit sequencing of a target nucleic acid within a chromosome, such as a chromosome fixed to a glass slide, a chromosome within a cell, or a chromosome within a tissue sample or tissue slice.

In one aspect, provided herein is a method for obtaining sequence information from a target nucleic acid in combination with spatial information, for example, within a chromosome sequence. In some embodiments, the method comprises hybridizing a primer to one strand of a chromosome in situ and ligating a labeled nucleotide to one end of the hybridized primer, wherein the labeled nucleotide is complementary to a nucleotide, directly downstream from a termini of the hybridized primer, on the chromosome strand hybridized to the primer, and detecting incorporation of the labeled nucleotide into the hybridized primer.

In some embodiments, the method comprises ligating a labeled nucleotide to a terminus of a nicked strand of a chromosome in situ, wherein the labeled nucleotide is complementary to a nucleotide, directly downstream from the termini of the nicked strand, on a chromosome strand complementary to the nicked strand, and detecting incorporation of the labeled nucleotide into the nicked strand.

### Definitions

As used herein, the terms “nucleic acid” and “polynucleotide” are used interchangeably herein to refer to a polymer having multiple nucleotide monomers. A nucleic acid can be single- or double-stranded, and can be DNA (e.g., cDNA or genomic DNA), RNA, or hybrid polymers (e.g., DNA/RNA). Nucleic acids can be chemically or biochemically modified and/or can contain non-natural or derivatized nucleotide bases. “Nucleic acid” does not refer to any particular length of polymer or number of bases e.g., greater than about 2 bases, greater than about 10 bases, greater than about 100 bases, greater than about 500 bases, greater than 1000 bases, greater than 10,000 bases, greater than 100,000 bases, greater than about 1,000,000, up to about 1010 or more bases composed of nucleotides The term “polynucleotide” is intended to include polynucleotides comprising naturally occurring nucleotides and/or non-naturally occurring nucleotides.

The term “target nucleic acid” refers to a nucleic acid whose presence in a sample can be identified and sequenced.

As used herein, the term “biological sample” means any biological sample that comprises nucleic acid sequences including, but not limited to cDNA, mRNA, synthetic chromosome and genomic DNA. Exemplary biological samples include tissue samples, such as liver, spleen, kidney, lung, breast, pancreas, intestine, thymus, colon, tonsil, testis, skin, brain, heart, muscle, and pancreas tissue. Other exemplary biological samples include, but are not limited to, biopsies, bone marrow samples, organ samples, skin fragments and organisms. Materials obtained from clinical or forensic settings are also within the intended meaning of the term biological sample. In one embodiment, the sample is derived from a human, animal or plant. In one embodiment, the biological sample is a tissue sample, preferably an organ tissue sample. In one embodiment, samples are human. The sample can be obtained, for example, from autopsy, biopsy, muscle punch, or from surgery. It can be a solid tissue or solid tumor such as parenchyme, connective or fatty tissue, heart or skeletal muscle, smooth muscle, skin, brain, nerve, kidney, liver, spleen, breast, carcinoma (e.g., bowel, nasopharynx, breast, lung, stomach etc.), cartilage, lymphoma, meningioma, placenta, prostate, thymus, tonsil, umbilical cord or uterus. The tissue can be a tumor (benign or malignant), cancerous or precancerous tissue. The sample can be obtained from an animal or human subject affected by disease or other pathology or suspected of same (normal or diseased), or considered normal or healthy.

The term “fixed biological sample” is used herein in a broad sense and is intended to include sources that contain nucleic acids and can be fixed. As used herein, the term “fixed biological sample,” excludes cell-free samples, for example cell extracts, wherein cytoplasmic and/or nuclear components from cells are isolated but not extra cellular DNA or cell free DNA.

As used herein, the term “isolated and fixed chromosome” or “chromosome spread” are used to refer to chromosomes that are substantially free of other cellular material and are fixed to a surface, such as a glass slide.

“Fixation” of a biological sample or isolated chromosome can be effected with fixatives known to the person skilled in the art. In one embodiment, the fixative includes but is not limited to, acids, alcohols, ketones or other organic substances, such as, methanol-acetic acid, glutaraldehyde, formaldehyde or paraformaldehyde. Examples of fixatives and uses thereof may be found in Sambrook et al. (2000) and Maniatis et al. (1989). In one embodiment, the used fixation also preserves DNA and RNA. In one embodiment, the fixed biological sample may or may not be embedded. Embedding materials include, but are not limited to, paraffin, mineral oil, non-water soluble waxes, celloidin, polyethylene glycols, polyvinyl alcohol, agar, gelatine, nitrocelluloses, methacrylate resins, epoxy resins or other plastic media. Thereby, one can produce tissue sections of the biological material suitable for histological examinations. In some embodiments, the fixed sample is permeabilized.

As used herein, the term “chromosome” refers to the support for the genes carrying heredity in a living cell, including DNA, protein, RNA and other associated factors. The conventional international system for identifying and numbering the chromosomes of the human genome is used herein. The size of an individual chromosome may vary within a multi-chromosomal genome and from one genome to another.

The term “intact chromosome” refers to a chromosome that contains a centromere, a long arm containing a telomere and a short arm containing a telomere.

The term “hybridization” refers to the specific binding of a nucleic acid to a complementary nucleic acid via Watson-Crick base pairing. In some embodiments, the term “hybridization” includes the specific binding of a nucleic acid to a complementary nucleic acid via non-canonical base pairing (e.g. Hoogsteen base pairing) or metal mediated base pairing. The terms “hybridizing” and “binding”, with respect to nucleic acids, are used interchangeably.

“Hybridizing specifically to” or “specifically hybridizing to” or like expressions refer to the binding, duplexing, triplexing, quadruplexing or hybridizing of a primer or MIP substantially to or only to a particular nucleotide sequence or sequences under stringent conditions. It is to be understood that any desired stringency and/or conditions can be employed as desired. The term “chromosomal region” as used herein denotes a contiguous length of nucleotides in a genome of an organism. A chromosomal region may be in the range of 10 kb in length to an entire chromosome, e.g., 100 kb to 10 MB for example.

The term “in situ hybridization” refers to hybridization of a primer or MIP that is complementary to specific nucleic acid sequence present in an intact chromosome, where the intact chromosome can be present inside a cell or is isolated from a cell.

The term “in situ hybridization conditions” as used herein refers to conditions that allow hybridization of a nucleic acid to a complementary nucleic acid in an intact chromosome. Suitable in situ hybridization conditions can include both hybridization conditions and optional wash conditions, which include temperature, concentration of denaturing reagents, salts, incubation time, etc. Such conditions are known in the art.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages can mean±5% (e.g., ±4%, ±3%, ±2%, ±1%) of the value being referred to.

Where a range of values is provided, each numerical value between the upper and lower limits of the range is contemplated and disclosed herein.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), and Current Protocols in Protein Science (CPPS), John E. Coligan (ed.), John Wiley and Sons, Inc., 2005 (ISBN 0471142735), the contents of which are all incorporated by reference herein in their entireties.

### In Situ Sequencing

In situ sequencing (ISS) methods are those by which a target nucleic acid, such as genomic DNA or mRNA, is sequenced directly while retained in a section of fixed tissue, a fixed cell sample or in fixed isolated chromosomes. That is, in situ sequencing does not require isolation of nucleic acids from a biological sample prior to sequencing. Rather, methods for in situ sequencing as described herein link sequencing information and its spatial location.

Certain methods of in situ sequencing permit the quantification of multiple mRNA sequences simultaneously and also permit spatial information with single-cell resolution. For example, the in situ sequencing method described in Ke et al. (In situ sequencing for RNA analysis in preserved tissue and cells Nature Methods 10, 857-860 (2013)) uses four fluorescent dyes to indicate nucleic acid bases, padlock probes for RNAs of interest, and enzymes that catalyze the formation of circularized DNA at the locations of the padlock probes, in a mechanism called rolling circle amplification. The resulting fluorescent DNAs are read using imaging technologies. This method permits the generation of mRNA expression profiles with location information within tissues.

Another method of in situ sequencing is hybridization-based in situ sequencing (HybISS), which uses rolling circle amplification of padlock probes. The padlock probe design permits the use of a barcoding approach that uses sequencing-by-hybridization chemistry. An alternative untargeted approach known as fluorescent in situ sequencing (FISSEQ) is performed in preserved tissue and permits single molecule in situ RNA localization. The FISSEQ method uses a nucleic acid sequencing library construction method that stably cross-links cDNA amplicons within biological samples. Sequencing data is then generated through an intensive interleaved microscopy and biochemistry protocol and subsequent image processing and bioinformatics.

Provided herein is an alternative method of in situ sequencing that can permit sequencing of a target nucleic acid directly from an intact, nicked, or otherwise relaxed chromosome.

### Samples Comprising Target Nucleic Acids

The methods described herein can be performed on a variety of biological or clinical samples that comprise a target nucleic acid in which spatial information and sequence information are desired. Typically, such biological or clinical samples contain cells comprising chromosomes, but the term can also refer to non-cellular or isolated biological material, such as spread chromosomes. Exemplary biological samples include, but are not limited to, tissue biopsies, scrapes (e.g., buccal scrapes), blood, plasma, serum, urine, saliva, cell culture, tumor samples, organ samples, skin samples, cerebrospinal fluid, animal or plant tissue, peripheral blood lymphocytes, touch preparations prepared from uncultured primary tumors, cancer cells, bone marrow, cells obtained from biopsy, cells from amniotic fluid, cells from maternal blood (e.g., fetal cells), cells from testis and ovary, A biological sample can comprise a tissue or fluid sample obtained from an individual including, but not limited to, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including, but not limited to, blood cells), tumors, organs, and also samples of in vitro cell culture constituent. In some embodiments, the sample is from a resection, bronchoscopic biopsy, or core needle biopsy of a primary or metastatic tumor. In addition, fine needle aspirate samples can be used. Samples can be either embedded, for example, in paraffin.

Typically, the target nucleic acid is retained in a chromosomal structure, which can be isolated and fixed on a slide, or the chromosome can be present in a cell or population of cells that are in any (or all) stage(s) of the cell cycle (e.g., mitosis, meiosis, interphase, G0, G1, S and/or G2). Analytical samples can be prepared using conventional techniques, which typically depend on the biological source from which a biological sample or specimen is taken. Essentially any cell or tissue obtained from a living organism having DNA can be sequenced using the methods and compositions described herein. Examples of such DNA sources described herein are not to be construed as limiting the sample types applicable to the methods described herein.

The methods described herein can be used to obtain chromosome sequence information in multiple settings, e.g., live cells, fixed cells, chromosome spreads (e.g., metaphase chromosome spread or fixed isolated chromosomes), chromatin fiber, and/or fixed tissue (e.g., formalin-fixed, paraffin-embedded tissue), and/or blood or bone marrow smear. Accordingly, in some embodiments, the chromosome is present in a cell. It is noted the cell can be present in a tissue or section thereof. In some embodiments, the cell, tissue or section thereof can be present in a matrix.

In some embodiments, the chromosome is a spread chromosome (e.g., an isolated and fixed chromosome on a glass slide) or stretched chromatin fiber.

### Primers

As used herein, the term “primer” means an oligonucleotide, either natural or synthetic, which is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Primers can be extended by a DNA polymerase. Primers for use with the methods and compositions provided herein can have any desired nucleotide length and nucleic acid sequence. Generally, a primer comprises between about 10 nucleotides to about 100 nucleotides, between about 10 nucleotides to about 70 nucleotides, between about 15 nucleotides to about 50 nucleotides, between about 20 nucleotides to about 60 nucleotides and all ranges and values in between whether overlapping or not.

The primer comprises a nucleotide sequence complementary to a strand of a chromosome (e.g., a repetitive element). Such a sequence is also referred to as a “complementary sequence,” a “target nucleic acid,” or a “target hybridizing sequence” herein. The target hybridizing sequence can be between about 10 nucleotides to about 50 nucleotides, between about 10 nucleotides to about 40 nucleotides, between about 15 nucleotides to about 30 nucleotides, between about 20 nucleotides to about 25 nucleotides and all ranges and values in between whether overlapping or not. For example, the target hybridizing sequence can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

It is noted that the primer can be any sequence that can bind to the genome. For example, the primer can comprise a mixture of random bases (e.g. N [mixture of A, C, T, G], deoxyinosine [universal base], etc.). In some embodiments of any one of the aspects, the primer can comprise only one type of nucleobase. For example, the primer can comprise all T's, e.g., for binding to polyA sequences.

The primers can be designed to hybridize anywhere on the chromosome. For example, primers can be designed to hybridize with a repetitive element, a transposable element, a regulatory element, a telomere, and/or a centromere. In some embodiment, the primer hybridizes to a repetitive element. As used herein, the term “repetitive element” refers to a DNA sequence that is present in many identical or similar copies in the genome. Repetitive elements include a DNA sequence that is present on each copy of the same chromosome (e.g., a DNA sequence that is present only once, but is found on both copies of a chromosome would be considered a repetitive element). In some embodiments, the repetitive elements do not comprise a tandem repeat (i.e., a sequence that is repeated directly following the end of the prior repeat such as the following tandem repeat of the sequence GATC: GATCGATCGATC etc).

In embodiments where a primer is designed to bind to an interspersed repetitive nucleic acid sequences, the primer can bind to an Alu repeat or a Line (L1) repeats Alu repeats are the most abundant interspersed repetitive nucleic acid sequence in the human genome with a total copy number of approximately 1 million. The Alu sequence is approximately 300 base pairs in length and occurs with an average frequency of once every 3300 base pairs. They occur throughout the primate family and are homologous to a small, abundant RNA gene that codes for the 300-nucleotide-long RNA molecule known as 7SL. L1 repetitive nucleic acid sequences are interspersed repeat sequences of between 1000 and 7000 base pairs. Lis have a common sequence at the 3′ end, but are variably shortened at the 5′ end (accounting for the disparity in length). They occur on average every 28,000 base pairs in the human genome, for a total copy number of about 100,000. Unlike Alu repetitive nucleic acid sequences, which are restricted to primates, L1 repetitive nucleic acid sequences are found in most other mammalian species.

Microsatellite repeats include a variety of simple di-, tri-, tetra-, and penta-nucleotide tandem repeats that are dispersed in the euchromatic arms of most chromosomes. The dinucleotide repeat (GT)n is the most common of these dispersed repetitive nucleic acid sequences, occurring on average every 30,000 bases in the human genome, for a total copy number of 100,000. The GT repeats range in size from about 20 to 60 base pairs and appear in most eukaryotic genomes. Minisatellite repeats are a class of dispersed tandem repeats in which the repeating unit is 30 to 35 base pairs in length and has a variable sequence, but contains a core sequence 10 to 15 base pairs in length. Minisatellite repeats range in size from 200 base pairs up to several thousand base pairs, and are present in lower copy numbers than microsatellite repeats. Minisatellite repeats tend to occur in greater numbers toward the telomeric ends of chromosomes.

Other repetitive nucleic acid sequences are predominantly limited to particular structures of the chromosome. Telomere repeats consist of tandem repeats of the sequence TTAGGG and are located at the very ends of the linear DNA molecules in human and vertebrate chromosomes. Sub-telomeric repeats include classes of repetitive sequences that are interspersed in the last 500,000 bases of non-repetitive DNA located adjacent to the telomere. Some repetitive nucleic acid sequences are chromosome specific and others appear to be present near the ends of all human chromosomes.

Alpha satellite DNA is a family of related repetitive nucleic acid sequences that occur as long tandem arrays at the centromeric region of all human chromosomes. The repeat unit is about 340 base pairs, and appears as a dimer made up of two subunits, each about 170 base pairs long. Alpha satellite DNA occurs on both sides of the centromeric constriction and extends up to 5000 base pairs from the centromere.

Satellite I, II, and III repeats are the three classical human satellite DNAs. Satellite DNAs can be isolated from the bulk of genomic DNA by centrifugation in buoyant density gradients because their densities differ from the densities of other DNA sequences. Satellite I is rich in As and Ts and is composed of alternating arrays of a 17- and 25-base-pair repeating unit. Satellites II and III are both derived from the simple five-base repeating unit ATTCC. Satellite II is more highly diverged from the basic repeating unit than Satellite III. Satellites I, II and III occur as long tandem arrays in the heterochromatic regions of human chromosomes 1, 9, 16, 17, and Y and the satellite regions on the short (p) arms of human chromosomes 13, 14, 15, 21, and 22.

As noted above, the methods and compositions described herein can include methods for sequencing from repetitive elements by using a primer that binds to a given repetitive element. Without wishing to be bound by a theory, this can allow genome-wide sequencing using a small subset of primers directed to common (repetitive) sequences found in the genome. For example, a primer comprising a nucleotide sequence complementary to a repetitive element sequence can hybridize at the same repetitive element sequence at different locations on the genome. Incorporation of the labeled nucleotide, as described herein, provide sequence information around the repetitive element location where the primer is hybridized. As such, variations around the repetitive elements at various locations can be obtained simultaneously.

In some embodiments, the primer can comprise, in addition to the target hybridizing sequence, a nucleotide sequence that is not complementary to a chromosome. For example, the primer can comprise a barcode sequence. As used herein, the term “barcode sequence” refers to a unique nucleotide sequence that allows a corresponding nucleic acid base and/or nucleic acid sequence to be identified. Generally, barcode sequences can each have a length within a range of from about 5 nucleotides to about 40 nucleotides. For example, barcode sequences can each have a length of from about 5 nucleotides to about 35 nucleotides, from about 6 nucleotides to about 30 nucleotides, from about 7 nucleotides to about 25 nucleotides, from about 8 nucleotides to about 15 nucleotides. In some embodiments, barcode sequences can each have a length of 5, 6, 7, 8, 9, 11, 12, 13, 14, 15, 16, 17, 18, 19, 20, 21, 22, 23, 24, 25, 26, 27, 28, 29, 30, 31, 32, 33, 34, 34 or 35 nucleotides.

The barcode sequence can be present at the 5′-end of the target hybridizing sequence. In some embodiments, the barcode sequence can be present at the 3′-end of the target hybridizing sequence. The target hybridizing sequence and the barcode sequence can be linked directly to each other, i.e., there are no nucleotides present between the target hybridizing sequence and the barcode sequence. In some embodiments, at least one nucleotide can be present between the target hybridizing sequence and the barcode sequence. For example, 2, 3, 4, 5, 6, 7, 8, 9, 10, 11, 12, 13, 14, 15, 16, 17, 18, 19, 20 or more nucleotides can be present between the target hybridizing sequence and the barcode sequence.

It is noted that the barcodes can be interrogated using methods known to those of skill in the art including fluorescently labeled oligonucleotide/DNA/RNA hybridization, primer extension with labeled nucleotides, sequencing, e.g., sequencing-by-ligation, -synthesis or -hybridization. The barcodes can also be interrogated using ligated circular padlock probes as described in Larsson, et al., (2004), Nat. Methods 1:227-232, content of which is incorporated herein by reference in its entirety. Ligated circular padlock can be used to detect multiple primers in parallel, followed sequencing, e.g., sequencing-by-ligation, -synthesis or -hybridization of the barcode sequences in the padlock probe to identify individual primers

In some embodiments, the primer can comprise a label. For example, the primer can comprise a detectable label. The detectable label can be used to detect the primer hybridized to the chromosome. Thus, in some embodiments, the method further comprises detecting the primer hybridized to the chromosome.

In some embodiments, the primer can comprise a label, where the label is a functional group. For example, the label is a functional group for incorporating the primer into a matrix or linking with an element for detection. Some exemplary functional groups include, but are not limited to, an amino group, a N-substituted amino group, a carboxyl group, a carbonyl group, an acid anhydride group, an aldehyde group, a hydroxyl group, an epoxy group, a thiol, a disulfide group, an alkenyl group, an azide group, a diol group, a hydrazine group, a hydrazide group, a semicarbazide group, a thiosemicarbazide group, one partner of a binding pair, an amide group, an aryl group, an ester group, an ether group, a glycidyl group, a halo group, a hydride group, an isocyanate group, an urea group, an urethane group, and any combinations thereof.

In some embodiments of any one of the aspects, the method comprises detecting the primer hybridized to the chromosome strand. Such detection methods provide clear spatial information regarding the location(s) in which the primer hybridizes to the target nucleic acid in chromosome and 3D location of the chromosome in the sample.

### Molecular Inversion Probe (MIP)

Molecular inversion probes (also known in the art as “padlock probes”) are single-stranded DNA molecules that are conventionally used for identifying or sequencing a single nucleotide polymorphism (SNP). Such MIPs are designed to contain two regions complementary to regions in a target DNA that flank a given nucleotide (e.g., SNP) in question. MIPs generally comprise sequences for one or more universal primers, which are separated by an endoribonuclease recognition site and a 20-nt tag sequence. When used in a method for identifying a single nucleotide polymorphism (SNP), the probes undergo a unimolecular rearrangement: they are (1) circularized by filling gaps with nucleotides corresponding to the SNP in four separate allele-specific polymerization (A, C, G, and T) and ligation reactions; (2) linearized in an enzymatic reaction. As a result, the MIPs become “inverted”. Generally, this step of a SNP identification assay is followed by PCR amplification. Further processing of the probes depends on specific assay.

As used herein, the term “molecular inversion probe (MIP)” refers to a nucleic acid probes that hybridizes to a target nucleic acid in a loop with the 5′ and 3′ ends abutting or separated in the target with a small gap (e.g., a given nucleotide). The gap of where the MIP binds is the nucleotide site that is to be sequence. The MIPs are typically designed to interrogate a target nucleotide in the gap using the high specificity of the DNA polymerase reaction. If provided with the appropriate dNTP, the polymerase can fill the gap between the MIP 5′ and 3′ ends. For example, if the target nucleic acid has an adenine “A” in the gap, the polymerase can fill the gap if provided with a complementary dTTP. The polymerase will add a “T” and fill the gap in the so called gap-fill reaction. With the gap filled, a ligase can close the remaining nick and circularize the MIP. MIP reaction products are typically detected after an amplification step, such as PCR using primer binding sites within the MIPs or rolling circle amplification, on a capture array.

### Detectable Labels for Primers or Labeled Nucleotides

The in situ sequencing methods described herein can make use of labeled primers and/or labeled nucleotides to be incorporated into a primer (e.g., a labeled or unlabeled primer). A labeled primer provides spatial information regarding where the primer hybridizes, for example, the location on the chromosome. The primers can also be allele specific. The labeled nucleotide serves to provide sequence information for a given nucleotide. When both a labeled primer and a labeled nucleotide are used, it is preferable that the primer and nucleotide are differentially labeled such that the signal from the primer can be distinguished from that of the nucleotide.

It is noted that, as used herein, the term “label” is not limited to a detectable label, but can also include any moiety which can carry out a particular or desired function. For example, detectability, such as by imaging, is just an exemplary function for a label.

In some embodiments, the label can be a detectable label. As used herein, the term “detectable label” means a moiety capable of producing a detectable signal. Detectable labels include any molecule or composition detectable by spectroscopic, photochemical, biochemical, immunochemical, electrical, electromagnetic, optical, chemical, mechanical or any other appropriate means. Suitable labels include light-absorbing dyes, fluorescent molecules, radioisotopes, nucleotide chromophores, enzymes, substrates, chemiluminescent moieties, bioluminescent moieties, mass labels, electron dense particles, magnetic particles, spin labels, charged groups, and the like. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). In some embodiments, the detectable label comprises biotin, amines, metals, metal nanoclusters (e.g., gold, silver, platinum, or copper), metal nanoparticles (e.g., gold, silver, platinum, or copper), anchoring molecules, quantum dotes, fluorescent polydots or acrydite. In some embodiments of any of the aspects, the detectable label comprises DNA origami structures (i.e., nanoscale folding of DNA to create non-arbitrary two- and three-dimensional shapes); see e.g., Rothemund, “Folding DNA to create nanoscale shapes and patterns”, Nature 440, 297-302 (2006). In some embodiments of any of the aspects, the detectable labels are detected using electron microscopy, fluorescence microscopy, dark field microscopy, or any combination thereof.

As used herein “detectable signal” refers to a signal that can detected by appropriate means. For example, the detectable signal can be a color, an intensity or brightness, fluorescence lifetime, fluorescence anisotropy, a color gradient, an intensity gradient, emission spectrum characteristics, absorption spectrum characteristics, energy transfer, mechanical force characteristics, light scattering and the like. In some embodiments, the detectable signal comprises a cumulative signal produced from a plurality of detectable labels (i.e., multiple copies of a given fluorophore or multiple oligos) attached to a given primer or nucleotide. Such methods can employ the use of an antibody or portion thereof, or a nanoparticle (e.g., gold nanoparticle) to carry the plurality of detectable labels and which are attached to a primer or nucleotide using, for example, a cleavable linker.

In one embodiment, the label is a “fluorophore,” which is a label that is capable of emitting light when in an unquenched form (e.g., when not quenched by another agent). The fluorescent moiety emits light energy (i.e., fluoresces) at a specific emission wavelength when excited by an appropriate excitation wavelength. Exemplary fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; JF 549, JF 646, JF 594, CF 488, CF 568, CF 647, CF 750, CF 660, SiR, HMSiR, Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP-Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodide (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodaminelsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used. In some embodiments, a combination of different fluorophores is used, for example, to distinguish between A, T, C, or G nucleotides (i.e., sequential fluorophores) and to reduce background noise.

Other exemplary detectable labels include chemiluminescent or bioluminescent markers (e.g., biotin, luciferase (e.g., bacterial, firefly, click beetle and the like), luciferin, aequorin, lucigenin, luminol, isoluminol, theromatic acridinium ester, imidazole, acridinium salt and oxalate ester), radiolabels (e.g., ³H, ¹²⁵I, ³⁵S, ¹⁴C, ³²P, and ³³P), and spectral calorimetric labels such as colloidal gold or colored glass or plastic (e.g., polystyrene, polypropylene, and latex) beads.

In some embodiments of any of the aspects described herein, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use as detectable labels include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

In some embodiments, the label can be a nucleic acid strand. For example, the labeled nucleotide is comprised in an oligonucleotide.

It is noted that the label can be attached to the nucleotide via a cleavable linker.

A cleavable linker is one which is sufficiently stable under a first set of conditions and can be cleaved to release the two parts the cleavable linker is holding together (e.g., an antibody and detectable labels from a nucleotide; a gold nanoparticle with detectable labels and a nucleotide). Generally, cleavable linkers are susceptible to cleavage agents, e.g., photo or UV irradiation, degradative molecules (e.g., enzymes and chemicals), pH, redox potential and the like. Accordingly, the label can be attached to the nucleotide via a photo-cleavable, chemically cleavable or enzymatically cleavable linker.

A linker serves to connect a detectable label with any portion of a nucleotide or nucleic acid. In certain embodiments, the linker is connected to a nitrogenous base portion of the nucleotide or nucleotide analog. The linker can be connected to any atom on of the nitrogenous base portion of the nucleotide or analog thereof. For example, the linker can be attached to N3 of the base when the base is thymine or uracil, attached to N4 of the base when the base is cytosine or adenine, attached to N1, N2, or O6 of the base when the base is guanine, attached to N5 when the base is ψ-uridine or attached to N7 of the base when the base is 9-deaza-G or 9-deaza-A.The linker can include any type of chemistry that upon contact with an activating agent results in cleavage of the linker to release the detectable label and also results in elimination of any portion of the linker that remains attached to the nucleotide analog, thereby producing the natural nucleotide. An exemplary linker is a linker that includes a disulfide bond. Additional linkers that can be used are Staudinger linkers. In certain embodiments, a cyclization reaction is used to eliminate the portion of the linker that remains attached to the nucleotide or nucleotide analog after cleavage.

The terms “reversible blocking group,” and “blocking moiety” when used in reference to a reversible terminator nucleotide refers to a chemical moiety attached to the nucleotide sugar (e.g., deoxyribose), usually at the 3′-O position of the sugar moiety, which prevents addition of a nucleotide by a polymerase at that position. A reversible blocking group can be cleaved by an enzyme (e.g., a phosphatase or esterase), chemical reaction, heat, light, sound, mechanical force etc., to provide a hydroxyl group at the 3′-position of the nucleoside or nucleotide such that addition of a nucleotide by a polymerase may occur.

In some embodiments, the labeled nucleotide comprises a detectable label. For example, the labeled nucleotide comprises one or more detectable labels permitting detection of the nucleotide at single molecule level once the nucleotide is incorporated onto the hybridized primer or nicked chromosome strand.

In some embodiments, the labeled nucleotide can be indirectly labeled with a detectable label. For example, the nucleotide comprises a moiety that permits a sandwich hybridization with an “agent” that specifically binds with the moiety and is capable of producing a detectable signal. In other words, the label is a moiety that permits specific binding of an agent capable of producing a detectable signal to the nucleotide. This can allow detection of the nucleotide by sandwich hybridization with the labeled agent directed to that moiety. Accordingly, in some embodiments, detecting the incorporated labeled nucleotide comprises contacting the moiety with the agent that specifically binds to the moiety to produce a detectable signal.

In some embodiments, the agent comprises a detectable label. For example, the agent comprises one or more detectable labels permitting detection of the agent, and permitting indirect detection of the labeled nucleotide.

The moiety attached to the labeled nucleotide and that permits specific binding of the agent can take any of a number of different forms. For example, in some embodiments, the moiety can be a first member of a binding pair and the agent can be a second member of a binding pair. A “binding pair” refers to first and second molecules or functional groups that specifically bind to each other. Exemplary coupling molecule pairs include, without limitations, any haptenic or antigenic compound in combination with a corresponding antibody or binding portion or fragment thereof (e.g., digoxigenin and anti-digoxigenin antibody; mouse immunoglobulin and goat antimouse immunoglobulin) and non-immunological binding pairs (e.g., biotin-avidin, biotin-streptavidin), hormone (e.g., thyroxine and cortisol-hormone binding protein), receptor-receptor agonist, receptor-receptor antagonist, IgG-protein A, lectin-carbohydrate, enzyme-enzyme cofactor, enzyme-enzyme inhibitor, and complementary oligonucleotide pairs capable of forming nucleic acid duplexes).

In some embodiments, the moiety is a first nucleic acid strand and the agent is a second nucleic acid strand having a nucleotide sequence substantially complementary to a nucleotide sequence of the first nucleic acid stand.

In some embodiments, the moiety can be a hapten or an antigen.

In some embodiments, the agent that binds to the moiety is an antibody or a nanobody/single domain antibody. The term “antibody” is used herein in the broadest sense and covers fully assembled antibodies, antibody fragments which retain the ability to specifically bind to the antigen (e.g., Fab, F(ab′)2, Fv, and other fragments), single chain antibodies, diabodies, antibody chimeras, hybrid antibodies, bispecific antibodies, humanized antibodies, and the like.

In some embodiments, the agent is conjugated with a docking nucleic acid strand. Without wishing to be bound by theory, an agent conjugated with a docking strand can permit the use of super-resolution detection modalities such as such as Point Accumulation for Imaging in Nanoscale Topography (PAINT, DNA-PAINT), Stimulated Emission Depletion Microscopy (STED), Reversible Saturable Optical Fluorescence Transitions (RESOLFT), Stochastic Optical Reconstruction Microscopy (STORM, dSTORM), Photoactivated Localization Microscopy (PALM), Blink Microscopy (BM), and any other form of super-resolution microscopy.

In most super-resolution implementations, fluorophores are switched between fluorescence ON- and OFF-states, so that individual molecules can be localized consecutively. In methods relying on targeted readout schemes such as in Stimulated Emission Depletion Microscopy (STED) or other Reversible Saturable Optical Fluorescence Transitions (RESOLFT) techniques, fluorescence emission is actively confined to an area below the diffraction limit. Switching of fluorescent molecules can also be carried out stochastically such as in (direct) Stochastic Optical Reconstruction Microscopy (STORM, dSTORM), Photoactivated Localization Microscopy (PALM) and Blink Microscopy (BM) where most fluorescent molecules are prepared in a dark state and only stochastically switched on to emit fluorescence. In Point Accumulation for Imaging in Nanoscale Topography (PAINT), fluorescence switching is obtained by targeting a surface with fluorescent molecules. In all stochastic approaches, fluorescence from single molecules is localized in a diffraction-limited area to yield super-resolved images. DNA-PAINT uses transient binding of fluorescently labeled reporter strand (imager strands) to complementary docking strands to obtain switching between a fluorescence ON- and OFF-state, which is necessary for localization-based super-resolution microscopy.

In some embodiments, the agent is conjugated with a docking nucleic acid strand and the method comprises a step of producing an amplicon, i.e., amplifying the docking strand and detecting the amplicon.

Certain embodiments of the various aspects described herein include a step of amplifying a nucleic acid strand. Methods of amplifying nucleic acid sequences are well known in the art. Such methods include, but are not limited to, isothermal amplification, polymerase chain reaction (PCR) and variants of PCR such as multiplex RT-PCR, immuno-PCR, SSIPA, Real Time RT-qPCR and nanofluidic digital PCR. In some embodiments, the docking strand is amplified using an isothermal amplification. Non-limiting examples of isothermal amplification include, but are not limited to, Recombinase Polymerase Amplification (RPA), nested RPA, Loop Mediated Isothermal Amplification (LAMP), Helicase-dependent isothermal DNA amplification (HDA), thermophilic helicase-dependent amplification (tHDA), Nucleic acid sequence-based amplification (NASBA), strand displacement amplification (SDA), ligase chain reaction (LCR), nicking enzyme amplification reaction (NEAR), Polymerase Spiral Reaction (PSR), polymerase cross-linking spiral reaction (PCLSR), and transcription-based amplification systems (TAS) such as nucleic acid sequence based amplification (NASBA), Rolling Circle Amplification (RCA), and Rapid Amplification of cDNA Ends (RACE, “one-sided PCR”). In some embodiments, non-isothermal amplification methods can be used which include, but are not limited to, PCR, multiple displacement amplification (MDA), rolling circle amplification (RCA), ligase chain reaction (LCR), or ramification amplification method (RAM).

In some embodiments, the agent is conjugated with a docking nucleic acid strand and the method comprises a step of Signal Amplification By Exchange Reaction (SABER). SABER is described, for example, in J. Y. Kishi et al., Nature Methods 2019 and S. K. Saka et al. Nature Biotechnology 2019, contents of all of which are incorporated herein by reference in their entireties.

In some embodiments, the agent is conjugated with a docking nucleic acid strand and the method comprises hybridizing a reporter nucleic acid strand with the docking nucleic acid strand, wherein the reporter nucleic acid strand comprises a detectable label. For example, the reporter strand comprises one or more detectable labels.

In order to detect the presence of a complimentary nucleotide at a given spatial location, in some embodiments a labeled nucleotide is incorporated at the 3′ end of a primer that is designed to bind a target nucleic acid. Any detectable label can be added to the nucleotide provided that the signal of the label can be detected using, for example, a microscope, a spectrophotometer, a tube luminometer or plate luminometer, x-ray film, a scintillator, a fluorescence activated cell sorting (FACS) apparatus, a microfluidics apparatus or a combination thereof. As one of skill in the art will appreciate, the detection of a given nucleotide label will depend on the resolution or detection limit of a given detection method. In order to permit the production of a detectable signal, a plurality of detectable labels can be attached to a given nucleotide (e.g., using a cleavable linker) to permit amplification of the nucleotide signal from the sample.

In some embodiments, the labeled nucleotides used with the methods described are CoolMPS™ nucleotides (Drmanac, S et al. bioRxiv doi:10.1101/2020.02.19.953307; US2018/223358, the contents of which are incorporated herein by reference in their entirety).

### Incorporation of a Labeled Nucleotide

Embodiments of the methods described herein comprise incorporating a labeled nucleotide to a primer or a nicked chromosome strand. It is to be understood that incorporating a labeled nucleotide to a primer or a nicked chromosome strand means extending the primer or the nicked stand with the labeled nucleotide.

As used herein, “incorporation” in reference to incorporating or ligating a labeled nucleotide to a primer or a nicked chromosome strand means to form a covalent bond or linkage between the termini of a primer or nicked chromosome strand and a labeled nucleotide in a template-driven reaction. It is noted that the incorporation can be enzymatic or chemical.

Generally, incorporation reactions are carried out to form a phosphodiester linkage between a 5′-hydroxyl of a labeled nucleotide with a 3′-hydroxyl of a primer hybridized to a chromosome strand or a 3′-hydroxyl of a nicked chromosome strand. Alternatively, incorporation of a labeled nucleotide can include forming a phosphodiester linkage between a 3′-hydroxyl of the labeled nucleotide with a 5′-hydroxyl of a primer hybridized to a chromosome strand or a 3′-hydroxyl of a nicked chromosome strand.

In some embodiments of any one of the aspects, incorporation of a labeled nucleotide is carried out enzymatically. Template-driven incorporation reactions are well known in the art. See, for example, U.S. Pat. Nos. 4,883,750; 5,476,930; 5,593,826; 5,426,180; 5,871,921; U.S. Patent Pub. 2004/0110213; Xu and Kool, Nucleic Acids Res. (1999) 27:875; Higgins et al., Methods in Enzymol. (1979) 68:50; and Engler et al., The Enzymes, (1982) 15:3 (1982), contents of all of which are incorporated herein by reference in their entireties.

In some embodiments of any one of the aspects, incorporation of a labeled nucleotide occurs in presence of a polymerase, e.g., a DNA polymerase. Polymerases include those known to those of skill in the art useful for extending primers. In some embodiments, particularly where the method uses nicked DNA, the DNA polymerase used in the incorporation step is a strand-displacing polymerase. The term strand displacement describes the ability to displace downstream nucleic acid encountered during synthesis. Exemplary strand-displacing DNA polymerases include, but are not limited to, Polymerase I Klenow fragment, Bst polymerase, Phi-29 polymerase, and Bacillus subtilis Pol I (Bsu) polymerase.

In some embodiments of any one of the aspects, incorporation of a labeled nucleotide is produced by enzymatic ligation in presence of a ligase. Exemplary ligases include, but are not limited to, T4 DNA ligase, T7 DNA ligase, E. coli DNA ligase, Taq ligase, Pfu ligase and the like.

In some embodiments, the labeled nucleotide to be incorporated comprises a non-natural, modified, or synthetic nucleotide. A nucleic acid can also include nucleobase modifications or substitutions (e.g., “base modification” or “base substitution”). As used herein, “unmodified” or “natural” nucleobases include the purine bases adenine (A) and guanine (G), and the pyrimidine bases thymine (T), cytosine (C) and uracil (U), whereas “modified nucleobases” can include the synthetic and natural nucleobases including but not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. In some embodiments, nucleobases are selected for their ability to increase the binding affinity of the primer to the target nucleic acid sequence. Such nucleobases include 5-substituted pyrimidines, 6-azapyrimidines and N-2, N-6 and 0-6 substituted purines, including 2-aminopropyladenine, 5-propynyluracil and 5-propynylcytosine. As but one example, 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278, which can be further combined with 2′-O-methoxyethyl sugar modifications. In some embodiments of the methods described herein, a “modified nucleobase” can include d5SICS and dNAM, which are non-limiting examples of unnatural nucleobases that can be used separately or together as base pairs (see e.g., Leconte et. al. J. Am. Chem. Soc. 2008, 130, 7, 2336-2343; Malyshev et. al. PNAS. 2012. 109 (30) 12005-12010). In some embodiments, nucleic acids as described herein (e.g., oligonucleotide tags, readout molecules, secondary oligonucleotides, and/or primers) comprise one or more modified nucleobases known in the art, i.e., any nucleobase that is modified from an unmodified and/or natural nucleobase.

Exemplary conditions for the extension step include, but are not limited to, those described, for example, in Nguyen et. al. 2020 (DOI: 10.1038/s41592-020-0890-0) and Guo et. al. 2008 (DOI: 10.1073/pnas.0804023105), contents of both of which are incorporated herein by reference in their entireties. Exemplary conditions for the ligation step include, but are not limited to, those described, for example, in Nguyen et. al. 2020 (DOI: 10.1038/s41592-020-0890-0) and Mitra et. al. 2003. (DOI: 10.1016/s0003-2697(03)0029-4), contents of both of which are incorporated herein by reference in their entireties.

### Hybridization of Primer or Molecular Inversion Probe (MIP)

Hybridization of the primer with an incorporated labeled nucleotide or MIP to chromosome sequences can be accomplished by standard in situ hybridization (ISH) techniques. Generally, the primer with the incorporated labeled nucleotide or the MIP hybridizes to a portion of the chromosome that has separated into two strands. Accordingly, in some embodiments the method can comprise a pre-hybridization treatment to increase accessibility of the chromosomal sequences for hybridization with a primer or MIP, e.g., to generate a single stranded region on a chromosome.

Methods for increasing accessibility of chromosomal sequences for hybridization with nucleic acid probes are well known in the art. Such methods include, but are not limited to, denaturation with heat or alkali, UV radiation, endonuclease treatment and/or exonuclease treatment. For example, samples can be incubated with BrdU/BrdC before fixation. After incubation with BrdU/BrdC, single stranded regions for hybridization can be created on the chromosome by UV radiation and/or exonuclease treatment. In some embodiments, the sample can be irradiated to induce breaks in chromosome strands. The sample can also be treated with chemicals that damage DNA and cause breaks.

Generally, “hybridization conditions” will include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a primer or MIP will hybridize to its target sequence. Stringent conditions are sequence-dependent and, thus, are different in different circumstances. Longer fragments can require higher hybridization temperatures for specific hybridization. As other factors can affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the melting temperature (T_{M}) for the specific sequence at a defined ionic strength and pH. Exemplary stringent conditions can include salt concentrations from about 0.01 M to about 1 M at a pH from about 7.0 to about 8.3 and a temperature of at least 25° C. (e.g., at least 26° C., at least 27° C., at least 28° C., at least 29° C., at least 30° C. or more). For example, conditions of 5×SSPE (750 mM NaCl, 50 mM Na phosphate, 5 mM EDTA, pH 7.4) and a temperature of 25-30° C. are suitable. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1^{st} Ed., BIOS Scientific Publishers Limited (1999). Hybridization conditions are also described in U.S. Pat. No. 5,447,841, contents of which are incorporated herein by reference in their entireties.

### Sequencing-by-Synthesis

In some embodiments, the methods described herein use massively parallel sequencing methods such as sequencing-by-synthesis (SBS). SBS utilizes controlled (i.e., one at a time) incorporation of a complementary nucleotide opposite the oligonucleotide being sequenced. This allows for accurate sequencing by adding nucleotides in multiple cycles as each nucleotide residue is sequenced one at a time, thus preventing an uncontrolled series of incorporations occurring. In one approach, reversible terminator nucleotides (RTs) are used to determine the sequence of the DNA template. In the most commonly used SBS approach, each RT comprises a modified nucleotide that includes (1) a blocking group that ensures that only a single base can be added by a DNA polymerase enzyme to the 3′ end of a growing DNA copy strand, and (2) a fluorescent or other label that can be detected, e.g., by a camera. In the most common SBS methods, templates and sequencing primers are fixed to a solid support and the support is exposed to each of four DNA nucleotide analogs, each comprising a different fluorophore attached to the nitrogenous base by a cleavable linker, and a 3′-O-azidomethyl group at the 3′-OH position of deoxyribose, and DNA polymerase. Only the correct, complementary base anneals to the target and is subsequently incorporated at the 3′ terminus of the primer. Nucleotides that have not been incorporated are washed away and the solid support is imaged. TCEP (tris(2-carboxyethyl)phosphine) is introduced to cleave the linker and release the fluorophores and to remove the 3′-O-azidomethyl group, regenerating a 3′-OH. The cycle can then be repeated (Bentley et al., Nature 456, 53-59, 2008). A different fluorescent color label is used for each of the four bases, so that in each cycle of sequencing, the identity of the RT that is incorporated can be identified by its color. An analogous approach can be used for sequencing from a chromosome template, with modifications made as described herein to detect single nucleotide incorporation events.

### Nucleic Acid Polymerases

Nucleic acid polymerases generally useful in the in situ sequencing methods described herein include DNA polymerases, RNA polymerases, reverse transcriptases, and mutant or altered forms of any of the foregoing. DNA polymerases and their properties are described in detail in, among other places, DNA Replication 2nd edition, Kornberg and Baker, W. H. Freeman, New York, N.Y. (1991). Known conventional DNA polymerases useful in the compositions and methods described herein include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9° Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteriol, 127: 1550), DNA polymerase, Pyrococcus kodakaraensis KOD DNA polymerase (Takagi et al., 1997, Appl. Environ. Microbiol. 63:4504), JDF-3 DNA polymerase (from Thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma™ DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1I/DP2 DNA polymerase II (Cann et al, 1998, Proc. Natl. Acad. Sci. USA 95:14250).

Both mesophilic polymerases and thermophilic polymerases are contemplated. Thermophilic DNA polymerases include, but are not limited to, ThermoSequenase®, 9° Nm™ Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof. A highly-preferred form of any polymerase is a 3′ exonuclease-deficient mutant.

Reverse transcriptases useful in the methods and compositions described herein include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit. Rev Biochem. 3:289-347 (1975)).

### Detection Method

It is noted that detection of the nucleotide incorporated into a hybridized primer or a nicked chromosome strand or detection of a hybridized primer will depend on the particular detectable labels used. For example, the incorporated nucleotide or the hybridized primer can be detected using a microscope, a spectrophotometer, a tube luminometer or plate luminometer, x-ray film, a scintillator, a fluorescence activated cell sorting (FACS) apparatus, a microfluidics apparatus or the like.

In some embodiments of any of one of the aspects, the detectable label is a fluorophore or fluorescent compounds. Systems and devices for the measurement of fluorescence are well known in the art. Fluorescence measurement requires a light source that emits light comprising the appropriate absorption or excitation wavelength. The absorption or excitation wavelength of the compounds described herein is approximately 300-800 nm. In some embodiments of any of the aspects, the light source emits light comprising, consisting essentially of, or consisting of a wavelength of 300-870 nm. The light contacts the sample, which excites electrons in certain materials within the sample, also known as fluorophores, and causes the materials to emit light (light emission) in the form of fluorescence.

The system or device for measurement of fluorescence then detects the emitted light. In some embodiments, the system or device can comprise a filter or monochromator so that only light of desired wavelengths reaches the detector of the system or device. In some embodiments of any of the aspects, the system or device is configured to detect light comprising, consisting essentially of, or consisting of a wavelength of 300-800 nm. In some embodiments of any of the aspects, the system or device is configured to detect light comprising, consisting essentially of, or consisting of a wavelength of 300-800 nm. Suitable systems and devices are commercially available and can include, e.g., the 20/30 PV™ Microspectrometer or 508 PV™ Microscope Spectrometer from CRAIC (San Dimas, CA), the Duetta™, FluoroMax™, Fluorolog™, QuantaMaster 8000™, DeltaFlex™, DeltaPro, or Nanolog™ from Horiba (Irvine, CA), or the SP8 Lightning™, SP8 Falcon™, SP8 Dive™ TCS SPE™, HCS A™, or TCS SP8 X™ from Leica (Buffalo Grove, IL).

In some embodiments of any of the aspects, fluorescence photomicroscopy can be used. Alternatively, digital (computer implemented) fluorescence microscopy with image-processing capability can be used. For example, methods and systems known in the art for imaging fluorescence in situ hybridization (FISH) can be used. See, for example, Schrock et al. (1996) Science 273:494; Roberts et al. (1999) Genes Chrom. Cancer 25:241; Fransz et al. (2002) Proc. Natl. Acad. Sci. USA 99:14584; Bayani et al. (2004) Curr. Protocol. Cell Biol. 22.5.1-22.5.25; Danilova et al. (2008) Chromosoma 117:345; U.S. Pat. No. 6,066,459; and FISH TAG™ DNA Multicolor Kit instructions (Molecular probes), contents of all of which are incorporated herein by reference in their entireties.

In some embodiments of any one of the aspects, a nucleotide incorporated onto the hybridized primer or nicked strand is detected and image recorded using a computerized imaging system such as the Applied Imaging Corporation CytoVision™ System (Applied Imaging Corporation, Santa Clara, Calif.) with modifications (e.g., software, Chroma 84000 filter set, and an enhanced filter wheel). Other suitable systems include a computerized imaging system using a cooled CCD camera (Photometrics, NU200 series equipped with Kodak KAF 1400 CCD) coupled to a Zeiss Axiophot microscope, with images processed as described by Ried et al. (1992) Proc. Natl. Acad. Sci. USA 89:1388). Other suitable imaging and analysis systems are described by Schrock et al., supra; and Speicher et al. (1996) Nature Genet. 12:368.

In some embodiments of any of the aspects, a nucleotide incorporated onto the hybridized primer or nicked strand is detected and visualized with super resolution microscopy (e.g. Stochastic Optical Reconstruction Microscopy (STORM) Imaging).

### Quenching the Detectable Signal

In some embodiments of the methods described herein, the method comprises extinguishing, quenching or removing the detectable signal, for example, during a given round of sequencing. Methods for extinguishing, quenching or removing detectable signals are well known in the art. For example, a detectable signal can be bleached or digitally subtracted. In some embodiments, the label itself can be cleaved or removed and, optionally washed away.

### Iterative Sequencing

The in situ sequencing methods provided herein can comprise multiple rounds of sequencing, however the detectable signal from a first round may need to be extinguished prior to the next round of sequencing. Thus, in some embodiments, the method further comprises: (i) extinguishing or removing the detectable signal (e.g., during a first round of sequencing); (ii) incorporating a second or subsequent labeled nucleotide (optionally a different nucleotide than during the first or prior hybridization and extension reactions) onto the primer or the nicked strand; and (iii) detecting the newly incorporated nucleotide. It is noted that steps (i)-(iii), i.e., removing the detectable signal, adding a new nucleotide and detecting the incorporation of the new nucleotide can be repeated any desired number of times. For example, steps of removing the detectable signal, adding a new nucleotide and detecting the incorporation of the new nucleotide can be repeated 2, 3, 4, 5, 6, 7, 8, 9, 10, 11, 12, 13, 14, 15, 16, 17, 18, 19, 20 or more times to obtain additional sequence information. Thus, in some embodiments, the method further comprises (i) extinguishing or removing the detectable signal; (ii) incorporating a second or subsequent labeled nucleotide onto the primer or the nicked strand; (iii) detecting the newly incorporated nucleotide; and (iv) optionally repeating steps (i)-(iii) one or more times, for example, with a third, or fourth labeled nucleotide. The result of this process produces a series of images that, when stacked, produce the sequence of incorporations at each location on the chromosome.

### Uses

The methods described herein are useful for sequencing chromosomes and sub-chromosomal regions of chromosomes. The methods are applicable to sequencing chromosomes or sub-chromosomal regions during various phases of the cell cycle including, but not limited to, interphase, preprophase, prophase, prometaphase, metaphase, anaphase, telophase and cytokinesis.

In addition to simply obtaining nucleic acid sequence information for chromosomal sequences, the methods described herein provide the added benefit of retaining structural or location-specific information regarding the sequences detected. Without wishing to be bound by theory, the methods described herein can be used for detecting chromosome aberrations and/or abnormalities. As used herein, the terms “chromosomal aberration” or “chromosome abnormality” refer to a deviation between the structure of the subject chromosome or karyotype and a normal, i.e., non-aberrant homologous chromosome or karyotype. The deviation can be of a single base pair (e.g., a single nucleotide polymorphism) or of many base pairs. The terms “normal” or “non-aberrant,” when referring to chromosomes or karyotypes, refer to the karyotype or banding pattern found in healthy individuals of a particular species and gender. Chromosome abnormalities can be numerical or structural in nature, and include, but are not limited to, aneuploidy, polyploidy, inversion, translocation, deletion, duplication, fusion and the like. Chromosome abnormalities can be correlated with the presence of a pathological condition or with a predisposition to developing a pathological condition. Chromosome aberrations and/or abnormalities can also refer to changes that are not associated with a disease, disorder and/or a phenotypic change. Such aberrations and/or abnormalities can be rare or present at a low frequency, e.g., a few percent of the population such as polymorphic sequences.

In some embodiments, haplotype estimation (also known as “phasing”) of the sequenced genetic material can be used to assign alleles (the As, Cs, Ts and Gs) to the paternal and maternal chromosomes. Stated another way, haplotype estimation or phasing refers to the process of statistical estimation of haplotypes from genotype data. Numerous statistical methods for estimation of haplotypes are known to those of skill in the art and can be used with the methods described herein. Exemplary statistical methods for haplotype estimation include, but are not limited to SNPHAP, PHASE (Stephens, M. et al. (2001). The American Journal of Human Genetics 68 (4): 978-989), fastPHASE (Scheet, P.; Stephens, M. (2006). The American Journal of Human Genetics 78 (4): 629-644), BEAGLE (Browning, S. R.; Browning, B. L. (2007). The American Journal of Human Genetics 81 (5): 1084-1097), IMPUTE2 (Howie, B. N.; et al. (2009). Schork, Nicholas J (ed.). “A Flexible and Accurate Genotype Imputation Method for the Next Generation of Genome-Wide Association Studies”. PLOS Genetics. 5 (6): e1000529), MaCH (Li, Y. et al. (2010) Genetic Epidemiology 34 (8): 816-834), SHAPEIT1 (Delaneau, O. et al. (2011) Nature Methods 9 (2): 179-181), HAPI-UR (Williams, A. L. et al. (2012) The American Journal of Human Genetics 91 (2): 238-251) or SHAPEIT2 (Delaneau, O. et al. (2012) Nature Methods 10 (1): 5-6).

### In Situ Counting of Short Tandem Repeats Using Direct In Situ Sequencing

Only about five percent of human DNA is thought to code for traits. Most of the rest is made of long stretches of nucleotide base pairs whose function remains unclear. Within these stretches are short, moderately repetitive base pair sequences called short tandem repeats (STRs). The number of repeats in each stretch is inherited and is easily detected. This makes them ideal identifying markers within a person's genome.

Short tandem repeats are regions of DNA where a core repeat unit (2-7 nts) are tandemly repeated. STR copy number variation has direct links to neurodegenerative diseases (Huntington's, Fragile X, etc.) and has been used in forensics to identify samples. Current Next Generation Sequencing (NGS) approaches have difficulty counting these regions as their repetitive nature makes accurate assembly difficult. STRs are analyzed using PCR and electrophoresis methods, which are low plex, low throughput and cannot maintain any spatial tissue information.

Provided herein are methods to count Short Tandem Repeats (STRs) in situ at a single cell resolution, using chromosomeSEQ with ligation of specific oligonucleotides that can be detected. In one example, a ligation primer is hybridized directly adjacent to an STR region. The first STR unit is counted/sequenced by ligation of oligonucleotide complementary to one STR unit. Only one oligonucleotide is ligated and subsequently detected. This detection indicates that 1 unit of the STR is present. Oligonucleotide is cleaved, exposing 5′ phosphate, allowing for the next STR unit to be counted/sequenced, if present. The cycle of ligation, detection, and cleavage is repeated until an entire STR region is counted/sequenced. Absence of signal indicates the end of the STR region, as there is not an STR unit present for hybridization and ligation.

In some embodiments, the method for counting the STRs comprises: (i) hybridizing a first oligonucleotide directly adjacent to an STR region in a target nucleic acid; (ii) ligating a second oligonucleotide to the first oligonucleotide that is hybridized with the target, wherein the second oligonucleotide comprises a nucleotide sequence complementary to one STR unit and wherein the second oligonucleotide comprises a detectable label; (iii) detecting the detectable label; (iv) cleaving the ligated second oligonucleotide, e.g., with a nicking enzyme to release the detectable label from the ligated oligonucleotide; and (v) repeating steps (ii)-(iv), until a detectable label is not detected in step (iv).

In some embodiments, the method for counting the Short tandem repeats (STRs) in a target nucleic acid comprises: (i) hybridizing a first oligonucleotide directly adjacent to a 3′-end of an STR region in a target nucleic acid; (ii) ligating a second oligonucleotide to a 5′end of the hybridized first oligonucleotide to generate a ligated oligonucleotide, wherein the second oligonucleotide comprises at its 3′-end a nucleotide sequence complementary to one STR unit and wherein the second oligonucleotide comprises at its 5′-end a detectable label; (iii) detecting the detectable label; (iv) cleaving the ligated oligonucleotide, e.g., with a nicking enzyme to release the detectable label from the ligated oligonucleotide; and (v) optionally, repeating steps (ii)-(iv), if the detectable label is detected in step (iv).

In some embodiments, the method for counting the Short tandem repeats (STRs) in a target nucleic acid comprises: (i) hybridizing a first oligonucleotide directly adjacent to a 3′-end of an STR region in a target nucleic acid-optionally, the first oligonucleotide comprises a phosphate group at its 5′-end; (ii) ligating a second oligonucleotide by its 3′-end to a 5′-end of the hybridized first oligonucleotide to generate a ligated oligonucleotide, wherein the second oligonucleotide comprises at its 3′-end a nucleotide sequence complementary to one STR unit and wherein the second oligonucleotide comprises at its 5′-end a detectable label; (iii) detecting the detectable label; (iv) cleaving the ligated oligonucleotide, e.g., with a nicking enzyme to release the detectable label from the ligated oligonucleotide and to generate a phosphate group at the 5′-end of the ligated oligonucleotide; (v) ligating a third oligonucleotide by its 3′-end to a 5′-end of the ligated oligonucleotide comprising the phosphate group to generate a ligated oligonucleotide, wherein the third oligonucleotide comprises at its 3′-end a nucleotide sequence complementary to one STR unit and wherein the third oligonucleotide comprises at its 5′-end a detectable label; (vi) detecting the detectable label; and (vii) optionally, repeating steps (iv)-(vi) if the detectable label is detected in step (vi).

In some embodiments of the method for counting the STRs, the first oligonucleotide further comprises a barcode, a hybridization domain for a barcode, a primer sequence or a hybridization domain for a primer. For example, the first oligonucleotide comprises at its 3′-end a barcode, a hybridization domain for a barcode, a primer sequence or a hybridization domain for a primer.

It is noted that the method for counting the STRs can be in situ or ex situ. Accordingly, in some embodiments of the method for counting the STRs, the method is in situ. In some other embodiments of the method for counting the STRs, the method is ex situ.

STRs can be detected for the purpose of forensic applications, detecting telomeric ends (e.g., the sequence TTAGGG is repeated 100-1000× in telomeres), or detection of disease. Certain diseases, including Huntington's disease, Fragile X syndrome, and Friedrich's ataxia, among others, have been shown to have expansion of repeats. For example, a subject having Huntington's disease comprises CAG repeats of at least 36 repeats (e.g., 36-120), while a subject lacking Huntington's disease comprises CAG repeats in the number of 10-35.

Exemplary embodiments of the various aspects described herein can be described by one or more of the numbered embodiments:

Embodiment 1: A method of determining sequence information, and optionally positional information, on a chromosome in situ, the method comprising: hybridizing a nucleic acid primer to a strand of a chromosome in situ under conditions that permit extension of the hybridized primer by a polymerase; extending the hybridized primer in presence of a polymerase with a nucleotide to produce an extended hybridized primer, wherein the nucleotide is complementary to a nucleotide, directly downstream from the hybridized primer, on the chromosome strand hybridized to the primer, and wherein the nucleotide is conjugated with a moiety that permits detection of the nucleotide with an agent that specifically binds to the moiety and is capable of producing a detectable signal; and contacting the moiety with the agent that specifically binds to the moiety and producing a detectable signal, thereby detecting incorporation of the nucleotide onto the hybridized primer.

Embodiment 2: The method of Embodiment 1, wherein said moiety is an antigen or antibody.

Embodiment 3: The method of Embodiment 1 or 2, wherein the moiety is an antibody.

Embodiment 4: The method of any one of Embodiments 1-3, wherein the moiety is conjugated with the nucleotide via a cleavable linker.

Embodiment 5: The method of any one of the preceding Embodiments, wherein the moiety is conjugated with one or more nanoparticles comprising a fluorophore.

Embodiment 6: The method of any one of the preceding Embodiments, wherein the agent is conjugated with a detectable label.

Embodiment 7: The method of any one of the preceding Embodiments, wherein the agent is conjugated with a docking nucleic acid strand.

Embodiment 8: The method of any one of the preceding Embodiments, wherein the agent is conjugated with a docking nucleic acid strand conjugated to a nanoparticle.

Embodiment 9: The method of Embodiment 8, wherein said detecting comprises a step of producing an amplicon from the docking strand nucleic acid strand and detecting the amplicon.

Embodiment 10: The method of Embodiment 8, wherein said detecting comprises a step of producing a Signal amplification by Exchange Reaction (SABER) amplification from the docking strand nucleic acid strand and detecting the SABER amplified signal.

Embodiment 11: The method of Embodiment 8, wherein said detecting comprises hybridizing a reporter nucleic acid strand with the docking nucleic acid strand, wherein the reporter nucleic acid strand comprises a detectable label.

Embodiment 12: The method of any one of the preceding Embodiments, wherein the agent is an antibody or nanobody.

Embodiment 13: The method of any one of the preceding Embodiments, wherein the method further comprises a step of extinguishing the detectable signal.

Embodiment 14: The method of any one of the preceding Embodiments, wherein the method further comprises: extending the extended hybridized primer in presence of a polymerase with a second nucleotide, wherein the second nucleotide is complementary to a nucleotide directly downstream from the extended hybridized primer on the chromosome strand to which the primer is hybridized, and wherein the nucleotide is conjugated with a second moiety that permits detection of the nucleotide with a second agent that specifically binds to the second moiety and is capable of producing a detectable signal; and contacting the second moiety with the second agent that specifically binds to the moiety and producing a detectable signal, thereby detecting incorporation of the second nucleotide on to the extended hybridized primer.

Embodiment 15: The method of any one of the preceding Embodiments, wherein the primer comprises a detectable label.

Embodiment 16: The method of any one of the preceding Embodiments, wherein the primer comprises a barcode sequence.

Embodiment 17: The method of any one of the preceding Embodiments, wherein the method further comprises detecting hybridization of the primer on the chromosome.

Embodiment 18: The method of any one of the preceding Embodiments, wherein the primer hybridizes to a repetitive element.

Embodiment 19: The method of Embodiment 18, wherein the repetitive element is selected from the group consisting of a Long Interspersed Nuclear Element (LINE), Short Interspersed Nuclear Elements (SINE), SVA element, Alu element, centromeric repeat, trinucleotide repeat and a telomeric repeat.

Embodiment 20: The method of any one of the preceding Embodiments, further comprising, prior to hybridizing the nucleic acid primer, generating a single-stranded region on the chromosome in situ that permits hybridizing with the primer.

Embodiment 21: The method of any one of the preceding Embodiments, wherein the chromosome is in a cell.

Embodiment 22: The method of Embodiment 21, wherein the cell is in a tissue or section thereof.

Embodiment 23: The method of Embodiment 22, wherein the cell, tissue or section thereof is in a matrix.

Embodiment 24: A method of determining sequence information, and optionally positional information, on a chromosome in situ, the method comprising: providing a chromosome in situ that has a nicked strand, the nick leaving an extendable terminus; extending the extendable terminus of the nicked strand of the chromosome in the presence of a polymerase with a nucleotide, wherein the nucleotide is complementary to a nucleotide, directly downstream from the extendable terminus, on a chromosome strand complementary to the nicked strand, and wherein the nucleotide is conjugated with a moiety that permits detection of the nucleotide with an agent that specifically binds to the moiety and is capable of producing a detectable signal; and contacting the moiety with the agent that specifically binds to the moiety and producing a detectable signal, thereby detecting incorporation of the nucleotide onto the nicked strand.

Embodiment 25: The method of Embodiment 24, further comprising creating a nick on a strand of the chromosome prior to extending with the polymerase and the nucleotide.

Embodiment 26: The method of Embodiment 24 or 25, further comprising, prior to extending with the polymerase and the nucleotide, contacting the nicked strand with an exonuclease.

Embodiment 27: The method of any one of the preceding Embodiments, wherein the polymerase is a strand-displacing polymerase.

Embodiment 28: The method of any one of the preceding Embodiments, wherein said moiety is an antigen or antibody.

Embodiment 29: The method of any one of the preceding Embodiments, wherein the moiety is an antibody.

Embodiment 30: The method of any one of the preceding Embodiments, wherein the moiety is conjugated with the nucleotide via a cleavable linker.

Embodiment 31: The method of any one of the preceding Embodiments, wherein the moiety is conjugated with one or more nanoparticles comprising fluorophore.

Embodiment 32: The method of any one of the preceding Embodiments, wherein the agent is conjugated with a detectable label.

Embodiment 33: The method of any one of the preceding Embodiments, wherein the agent is conjugated with a docking nucleic acid strand.

Embodiment 34: The method of any one of the preceding Embodiments, wherein the agent is conjugated with a docking nucleic acid strand conjugated to one or more nanoparticles.

Embodiment 35: The method of Embodiment 34, wherein said detecting comprises a step of producing an amplicon from the docking strand nucleic acid strand and detecting the amplicon.

Embodiment 36: The method of Embodiment 34, wherein said detecting comprises a step of producing a SABER amplification from the docking strand nucleic acid strand and detecting the SABER amplified signal.

Embodiment 37: The method of Embodiment 34, wherein said detecting comprises hybridizing a reporter nucleic acid strand with the docking nucleic acid strand, wherein the reporter nucleic acid strand comprises a detectable label.

Embodiment 38: The method of any one of the preceding Embodiments, wherein the agent is an antibody or nanobody.

Embodiment 39: The method of any one of the preceding Embodiments, wherein the method further comprises a step of extinguishing the detectable signal.

Embodiment 40: The method of any one of the preceding Embodiments, wherein the method further comprises: further extending the nicked strand in presence of a polymerase with a second nucleotide, wherein the second nucleotide is complementary to a nucleotide, directly downstream from the extended nicked strand, on the chromosome strand complementary to the nicked strand, and wherein the second nucleotide is conjugated with a second moiety that permits detection of the nucleotide with a second agent that specifically binds to the moiety and is capable of producing a detectable signal; and contacting the second moiety with the second agent that specifically binds to the second moiety and producing a detectable signal, thereby detecting incorporation of the second nucleotide on to the nicked strand.

Embodiment 41: The method of any one of the preceding Embodiments, wherein the chromosome is in a cell.

Embodiment 42: The method of Embodiment 41, wherein the cell is in a tissue or section thereof.

Embodiment 43: The method of Embodiment 42, wherein the cell, tissue or section thereof is in a matrix.

Embodiment 44: A method of determining sequence information, and optionally spatial information, on a chromosome in situ, the method comprising: hybridizing a molecular inversion probe (MIP) to a strand of a chromosome in situ under conditions that permit extension of the MIP, wherein a first end of the MIP hybridizes to a first region of the chromosome strand and the second end of the MIP hybridizes to a second region of the same chromosome strand and wherein the first and the second regions are separated by at least one nucleotide; extending one end of the hybridized MIP by at least one nucleotide in presence of a polymerase with a nucleotide, wherein the nucleotide is complementary to a nucleotide, directly downstream from the first or second end of the hybridized MIP, on the chromosome strand hybridized to the MIP; ligating together the two ends of the hybridized, extended MIP; amplifying the ligated MIP to generate a template strand; and sequencing the template strand.

Embodiment 45: The method of Embodiment 44, wherein said amplifying the MIP comprises rolling circle amplification.

Embodiment 46: The method of Embodiment 44, wherein said amplifying the MIP comprises SABER amplification.

Embodiment 47: The method of any one of the preceding Embodiments, wherein the MIP comprises a barcode sequence.

Embodiment 48: The method of any of any one of the preceding Embodiments, wherein the MIP comprises a priming sequence.

Embodiment 49: The method of any one of the preceding Embodiments, wherein said sequencing the template strand is by a fluorescence-based sequencing method.

Embodiment 50: The method of any one of the preceding Embodiments, wherein said sequencing the template strand is sequencing by ligation.

Embodiment 51: The method of any one of the preceding Embodiments, wherein said sequencing the template strand is sequencing by hybridization.

Embodiment 52: The method of any one of the preceding Embodiments, wherein said sequencing the template strand is sequencing by synthesis.

Embodiment 53: The method of any one of the preceding Embodiments, wherein the MIP hybridizes to a repetitive element.

Embodiment 54: The method of Embodiment 53, wherein the repetitive element is selected from the group consisting of a Long Interspersed Nuclear Elements (LINE), Short Interspersed Nuclear Elements (SINE), SVA element, Alu element, centromeric repeat, trinucleotide repeat and a telomeric repeat.

Embodiment 55: The method of any one of the preceding Embodiments, further comprising, prior to hybridizing the MIP, generating a single-stranded region on the chromosome for hybridizing with the MIP.

Embodiment 56: The method of any one of the preceding Embodiments, wherein the chromosome is in a cell.

Embodiment 57: The method of Embodiment 56, wherein the cell is in a tissue or section thereof.

Embodiment 58: The method of Embodiment 57, wherein the cell, tissue or section thereof is in a matrix.

Embodiment 59: A method of determining sequence information, and optionally positional information, on a chromosome in situ, the method comprising: hybridizing a nucleic acid probe to a strand of a chromosome in situ, wherein the nucleic acid probe comprises a barcode sequence, a docking sequence, and a sequence complementary to a nucleotide sequence of the chromosome strand; hybridizing a molecular inversion probe (MIP) to the docking sequence of the probe under conditions that permit extension of the MIP, wherein a first end of the MIP hybridizes to a first region of the probe and the second end of the MIP hybridizes to a second region of the probe and wherein the first and the second regions are separated by at least one nucleotide; extending one end of the hybridized MIP by at least one nucleotide in presence of a polymerase with a nucleotide, wherein the nucleotide is complementary to a nucleotide, directly downstream from the first or second end of the hybridized MIP, on the probe; ligating together two ends of the hybridized, extended MIP; amplifying the ligated MIP to generate a template strand; and sequencing the template strand.

Embodiment 60: The method of Embodiment 59, wherein said amplifying the MIP comprises rolling circle amplification.

Embodiment 61: The method of Embodiment 59, wherein said amplifying the MIP comprises SABER amplification.

Embodiment 62: The method of any one of the preceding Embodiments, wherein said sequencing the template strand is by a fluorescence-based sequencing method.

Embodiment 63: The method of any one of the preceding Embodiments, wherein said sequencing the template strand is sequencing by ligation.

Embodiment 64: The method of any one of the preceding Embodiments, wherein said sequencing the template strand is sequencing by hybridization.

Embodiment 65: The method of any one of the preceding Embodiments, wherein said sequencing the template strand is sequencing by synthesis.

Embodiment 66: The method of any one of the preceding Embodiments, wherein the MIP hybridizes to a repetitive element.

Embodiment 67: The method of Embodiment 66, wherein the repetitive element is selected from the group consisting of a Long Interspersed Nuclear Elements (LINE), Short Interspersed Nuclear Elements (SINE), SVA element, Alu element, centromeric repeat, trinucleotide repeat and a telomeric repeat.

Embodiment 68: The method of any one of the preceding Embodiments, further comprising, prior to hybridizing the MIP, generating a single-stranded region on the chromosome for hybridizing with the MIP.

Embodiment 69: The method of any one of the preceding Embodiments, wherein the chromosome is in a cell.

Embodiment 70: The method of Embodiment 69, wherein the cell is in a tissue or section thereof.

Embodiment 71: The method of Embodiment 70, wherein the cell, tissue or section thereof is in a matrix.

Embodiment 72: The method any one of the preceding Embodiments wherein the primer comprises a random mixture of nucleotides, e.g., the primer comprises a random sequence.

Embodiment 73: The method of any one of the preceding Embodiments, wherein the primer comprises at least one universal nucleobase.

Embodiment 74: The method of any one of the preceding Embodiments, wherein the primer comprises a homopolymer sequence, e.g., polyN sequence, where N is the same nucleotide.

Embodiment 75: The method of any one of the preceding Embodiments, wherein the primer comprises a quencher molecule.

Embodiment 76: The method of any one of the preceding Embodiments, wherein the primer comprises a moiety/label for isolating or purifying the primer (e.g., the extended primer).

Embodiment 77: The method of any one of the preceding Embodiments, wherein the chromosome is outside of a cell (e.g. extracellular chromosome/DNA, cell-free DNA).

Embodiment 78: A method for counting the short tandem repeats (STRs) in a target nucleic acid, the method comprising: (i) hybridizing a first oligonucleotide directly adjacent to a STR region in a target nucleic acid; (ii) ligating a second oligonucleotide to the first oligonucleotide that is hybridized with the target, wherein the second oligonucleotide comprises a nucleotide sequence complementary to one STR unit and wherein the second oligonucleotide comprises a detectable label; (iii) detecting the detectable label; (iv) cleaving the ligated second oligonucleotide, e.g., with a nicking enzyme to release the detectable label from the ligated oligonucleotide; and (v) repeating steps (ii)-(iv), until a detectable label is not detected in step (iv).

Embodiment 79: The method of Embodiment 78, wherein the method is in situ.

Embodiment 80: The method of Embodiment 79, wherein the method is ex situ.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

The description of embodiments of the disclosure is not intended to be exhaustive or to limit the disclosure to the precise form disclosed. While specific embodiments of, and examples for, the disclosure are disclosed herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments disclosed herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples in no way should be construed as being further limiting.

### EXAMPLES

Acquiring in situ nucleotide resolution with spatial information requires a method that sequences directly off of the chromosome in situ. Provided herein are methods for producing such information (e.g., collectively referred to as ‘chromoSEQ’ herein). In order to permit phasing of individuals, a direct readout of DNA in situ is necessary to determine whether they exist on the same homologous chromosome. Additionally, chromoSEQ can serve as a diagnostic for e.g., neurodegenerative diseases involving tandem repeats where the number of repeats is indicative of disease status. chromoSEQ can also be used to identify individual cells in a tissue that harbor detrimental mutations, as the single cell and direct sequencing capabilities permit the yield of this information.

With the three complimentary methods outlined for chromoSEQ (1. primeSEQ, 2. nickSEQ, 3. mipSEQ), the genome in individual cells can be directly sequenced. With primeSEQ, using common sequences found all over the genome (thousands to millions of copies for LINES, SINES, centromeric repeats, telomeric repeats), one can use a small subset of primers to permit genome-wide sequencing. In combination with the advances in sequencing by synthesis methods proposed herein, sufficient signal for detection of single nucleotides is permitted in situ. nickSEQ also provides in situ sequencing genome-wide without the requirement of synthesizing millions of individual primers. mipSEQ presents a targeted sequencing by synthesis with oligo conjugates.

The methods provided herein solve the problems associated with current sequencing by synthesis (SBS) methods that are restrictive and have relatively low signal to noise. Increasing sequencing signal improves conventional next generation sequencing (NGS) where the chemistry occurs in flow cells as well as in situ sequencing methods. Improvements to signal also increase confidence of signal detection, decreases detection time, and can increase the number of sequencing reads. This is especially useful for in situ sequencing, as the biological environment brings with it sources of signal perturbation. The methods provided herein also permit users to tailor nucleotide and fluorophore detection to best suit their needs. The use of oligonucleotide sequences permits the use of super-resolution modalities such as DNA-PAINT, and STORM. Oligonucleotide sequences can enhance diffraction limited signal through multiple fluorophores, HCR, RCA, SABER, and DNA branching.

Previous methods for SBS have: 1. Attached the fluorophore directly to the nucleotide (e.g. Illumina NextSeq), or, 2. Attached a primary antibody directly to the nucleotide, using a secondary antibody attached to a fluorophore for detection. Such methods do not conventionally permit the choice of fluorophores, do not allow for DNA PAINT, and do not allow for direct amplification of signal. However, in one embodiment, the inventors have devised a method that attaches oligonucleotide sequences to the secondary. Without wishing to be bound by a theory, this allows detection using any one of the many different detection methods for detecting nucleic acids. In addition, a docking strand can be used, which recruits imager strands for DNA PAINT, or complementary oligos can be used and conjugated to any fluorophore. Oligonucleotides can be amplified via rolling circle amplification (RCA), HCR, SABER, etc. resulting in significant signal amplification. Each of these signal amplification methods facilitate the detection of single nucleotide incorporation events, thereby facilitating direct sequencing of chromosome templates. Combinations of such signal amplification methods are also specifically contemplated to further improve or expand upon the ability to detect single nucleotide incorporation events.

### Example 1: Methods for Region Specific or Genome-Wide In Situ Sequencing Directly Off of Chromosomes (primeSEQ)

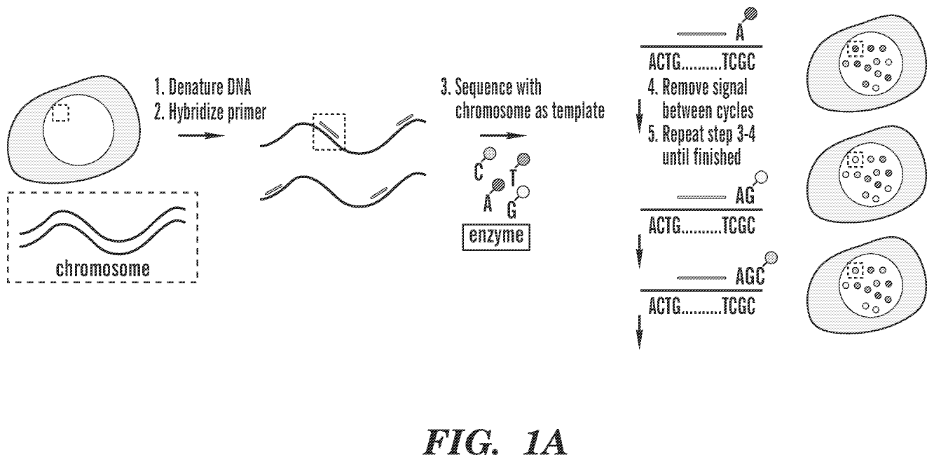

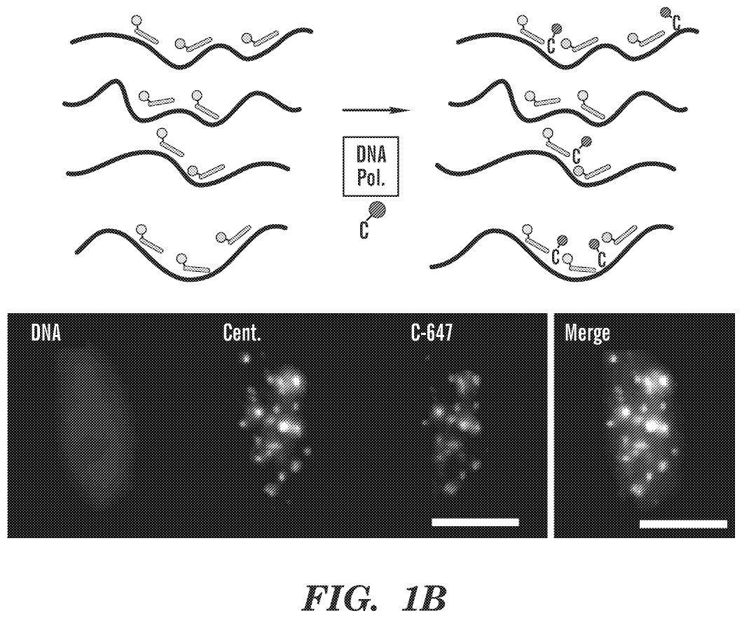

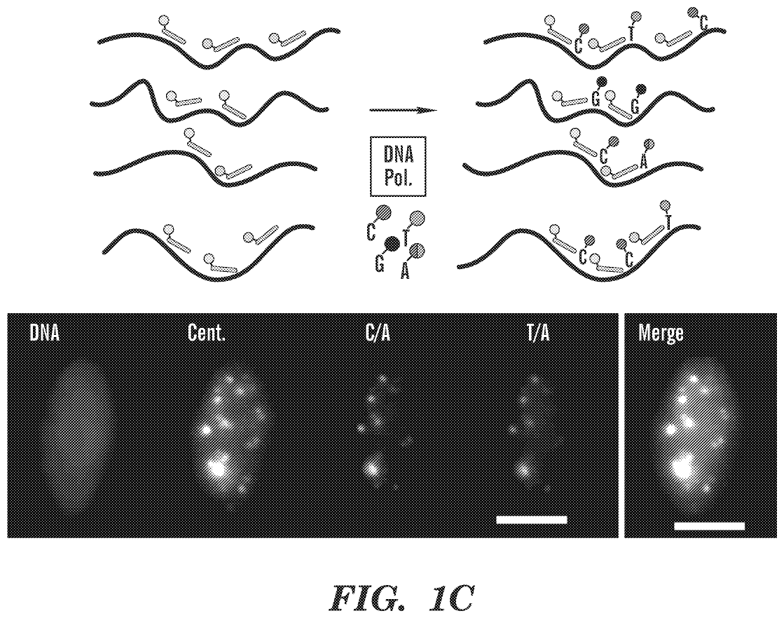

FIG. 1A is a schematic representation of an exemplary embodiment. Oligonucleotide primers (blue) are hybridized to genomic DNA after denaturation (1-2). Primers can be designed to any region and genome-wide coverage can be achieved in an economical manner using primers to repetitive regions (LINEs, SINEs, centromeric, telomeric, etc.) such that a common primer targeting consensus sequences is extended into unique sequences. The primer can contain a barcode that can aid in mapping. Primer can also be labeled directly with fluorophores, matrix attachment moieties for hydrogel embedding, elements for detection by mass spectrometry, gold nanoparticles for signal amplification. Primers are extended with in situ sequencing (ligation, synthesis, or hybridization) using the genome as a template. After sequencing, signal can be detected with conventional diffraction limited as well as super-resolution microscopy (3). Next, signal is removed (by, but not limited to photobleaching, enzymatic cleavage, light induced cleavage, chemical cleavage) (4). Extension/sequencing continues (5). After sequencing is complete, sequencing reads are mapped back.

FIG. 1B shows in situ sequencing directly off chromosomes according to an embodiment of the invention. Oligonucleotides (oligos) were hybridized to centromeric regions on multiple chromosomes. Oligos contained non-genome hybridizing regions that were visualized with a complementary oligonucleotide conjugated to a fluorophore (Alexa 488; green). Centromere targeting oligos were extended with DNA Polymerase and Alexa-647 labeled dCTP using the chromosome as a template. Here, dCTP does not contain a cleavable bond and signal is extinguished with photobleaching. Labeled nucleotides can be added individually or in combination with unlabeled nucleotides for sequence mapping.

FIG. 1C shows in situ sequencing directly off chromosomes according to an embodiment of the invention using Illumina NextSeq chemistry for 1 base of primer extension/se-quenching. Nucleotides have terminators that will allow only 1 base of extension. These nucleotides are cleavable and will remove signal as well as allow for the next base of extension/sequencing.

### Example 2: Methods for In Situ Sequencing Directly Off Single Stranded Genomic Regions Exposed by Exonuclease Treatment, DNA Damage, DNA Replication, and Chemical Treatment (expoSEQ)

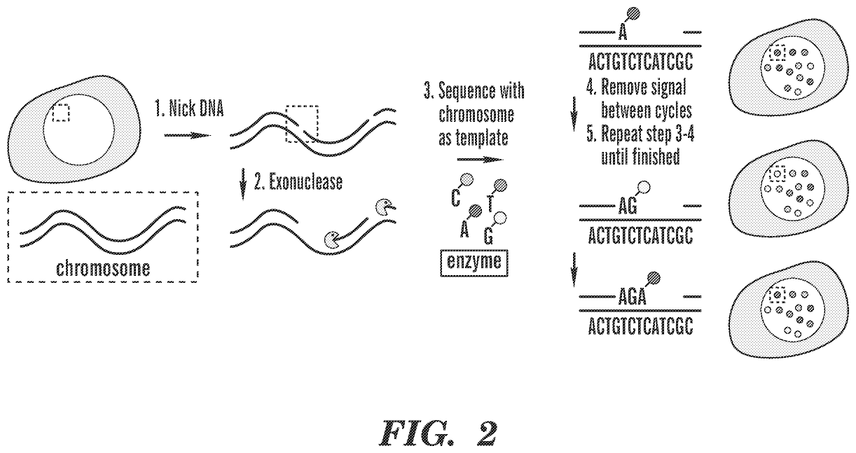

FIG. 2 is a schematic representation of another exemplary embodiment. Biological sample is treated with DNA nicking enzymes, forming single stranded breaks across the genome (1). Exonuclease treatment exposes larger regions of ssDNA through 3′ to 5′ or 5′ to 3′ resection. These resulting DNA will serve as a template for in situ sequencing (2). Digested and resected regions are extended with in situ sequencing (ligation, synthesis, or hybridization) using the genome as a template. Depicted here is sequencing by synthesis (3). After sequencing, signal can be detected with conventional diffraction limited as well as super-resolution microscopy (3). Next, signal is removed (by, but not limited to photobleaching, enzymatic cleavage, light induced cleavage, chemical cleavage) (4). Extension/sequencing continues (5). After sequencing is complete, sequencing reads are mapped back.

### Example 3: Methods for In Situ Sequencing Using Molecular Inversion Probes (mipSEQ)

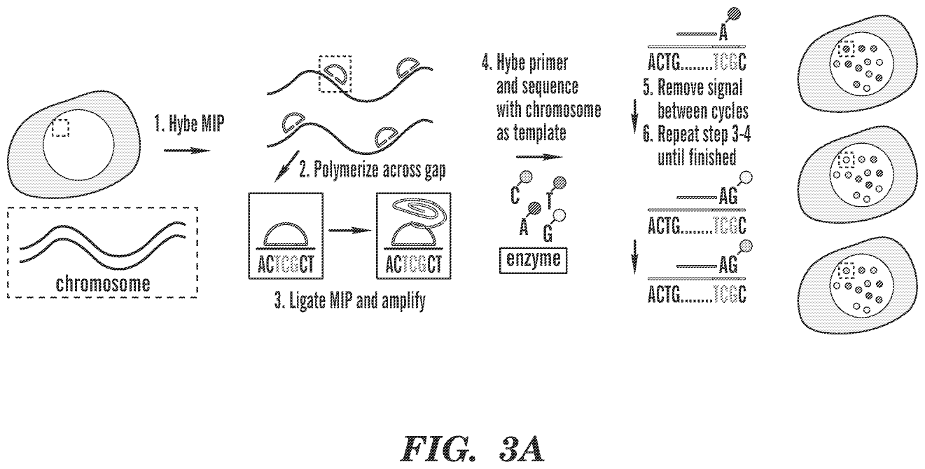

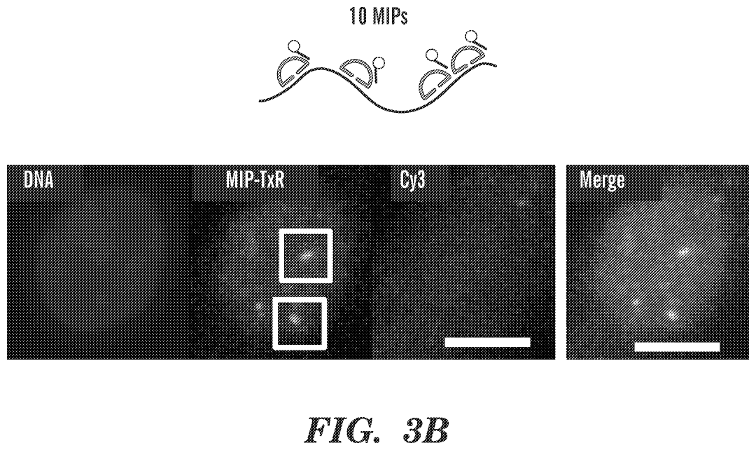

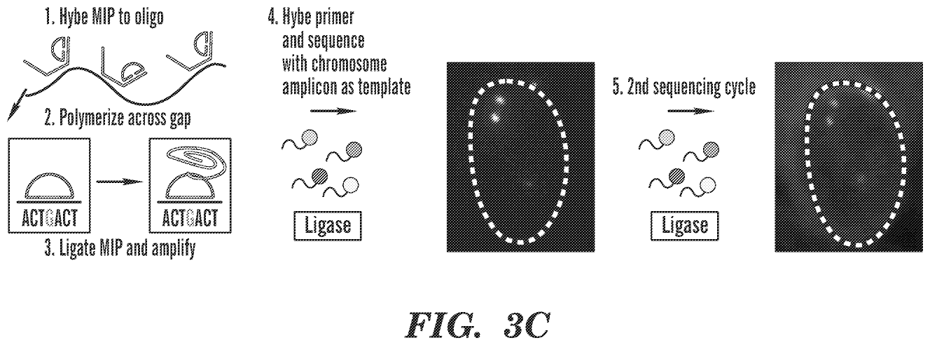

FIG. 3A is a schematic representation of another exemplary embodiment. Oligonucleotide molecular inversion probes (MIPs/padlock probes) are hybridized to genomic DNA after denaturation (1). MIPs can also contain barcodes to aid in location determination as well as to mediate the use of universal sequences for priming. Gaps are polymerized across with dNTPs and DNA polymerase, using genomic sequence as a template (2). After polymerizing across gaps, the MIP is circularized by ligation. Circularized MIPs can also be amplified through rolling circle amplification, producing a rolony, to enhance downstream signal (3). Circularized MIP or resulting rolony can be hybridized with an oligonucleotide primer and used for in situ sequencing (ligation, synthesis, or hybridization) to localize the MIP and identify nucleotides of the gap (4). Depicted here is sequencing by synthesis (SBS). After sequencing, signal can be detected with conventional diffraction limited as well as super-resolution microscopy (4). Next, signal is removed as described herein (5). Extension/sequencing continues (6). After sequencing is complete, sequencing reads are mapped back.

FIG. 3B shows hybridization and detection of 10 MIPs hybridized to chromosome in situ. MIPs were detected using a fluorophore conjugated oligonucleotide hybridizing to a barcode common to the backbone of all 10 MIPs. Absence of signal in Cy3 channel shown to confirm MIP detection signal. 10 μm scale bar.

FIG. 3C shows results of another exemplary embodiment. MIPs are hybridized to non-genome hybridizing region of oligonucleotide probe (Oligopaint) (1), 1 nucleotide gap polymerization (2), MIP circularization (3), MIP amplification by Rolling Circle Amplification (RCA) (3), and two cycles of in situ sequencing by ligation (4,5) to sequence across the gap.

### Example 4: Sequencing by Synthesis with Indirect Readout

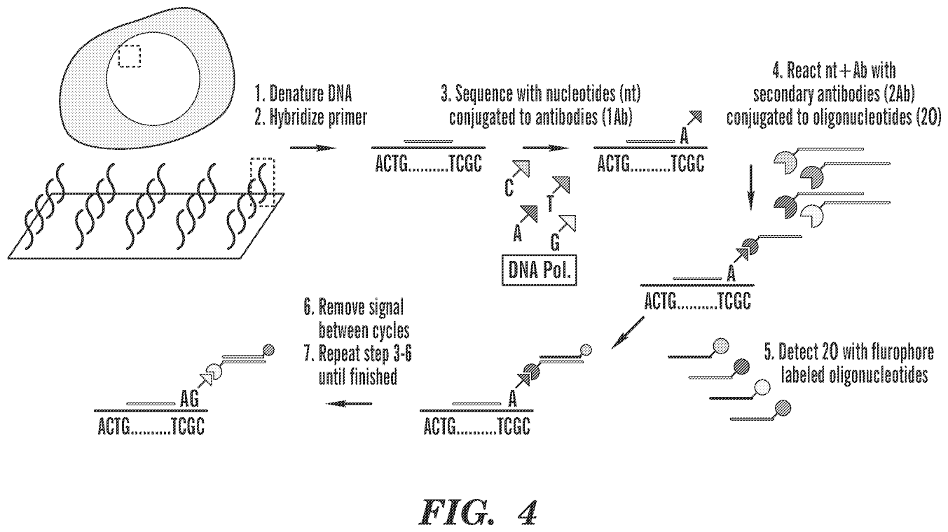

FIG. 4 is a schematic representation of an embodiment of a novel sequencing by synthesis based method using a three-part system. The method can be used for in situ as well as ex situ sequencing. DNA to be sequenced (examples may include chromosomes in a nucleus, barcodes on oligonucleotides (targeting genomic DNA, RNA, proteins), DNA on sequencing arrays, etc.) is hybridized with an oligonucleotide primer (blue) (1,2). Note that the primer can contain a barcode that will aid in mapping. Primer can also be labeled directly, e.g., with fluorophores, matrix attachment moieties for hydrogel embedding, elements for detection by mass spectrometry, gold nanoparticles for signal amplification. Sequencing by synthesis (SBS) is performed with antibody-conjugated reversible terminator nucleotides to extend primers. Each nucleotide (nt) is conjugated to a specific primary antibody (1Ab) (3). Specific secondary antibodies (2Ab) conjugated to oligonucleotides (20) react to specific antibodies on nucleotides (4). Secondary oligonucleotide sequences can also function as “docking strands” as used in DNA-PAINT methods for super-resolution imaging. Labeled oligonucleotides hybridize to and detect oligos on secondary antibodies (5). The labeled oligonucleotides may be imager strands for DNA-PAINT, labeled with multiple fluorophores to enhance signal, labeled with elements for detection by mass spectrometry, or recruit additional oligonucleotides for signal amplification (HCR, SABER, RCA). Signal can be detected with conventional diffraction limited as well as super-resolution microscopy (5). Next, signal is removed (by, but not limited to photobleaching, enzymatic cleavage, light induced cleavage, chemical cleavage) and a hydroxyl group is exposed for the next base to be sequenced (6). The rest of the sequence is read in the same manner (7).

### Example 5: Phasing by In Situ Sequencing and/or Homolog Specific Oligopaints (HOPs)

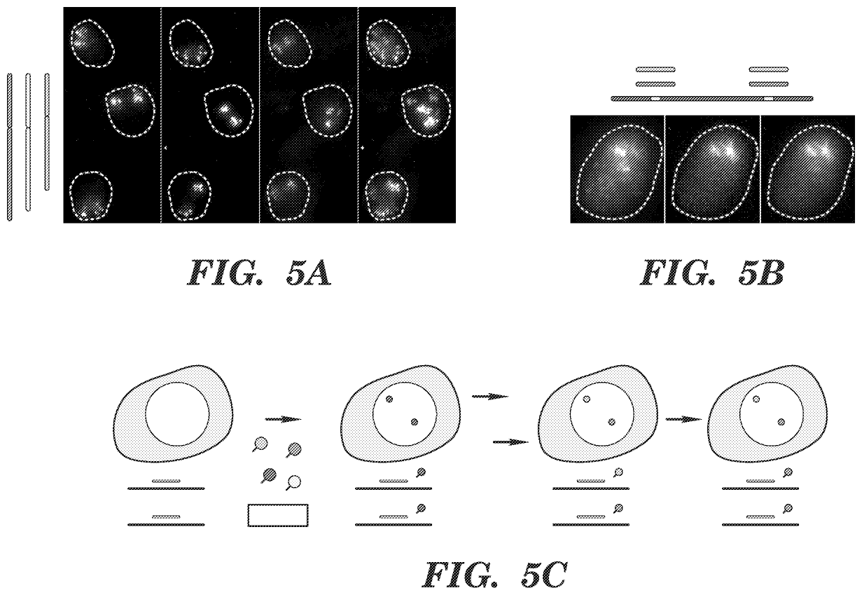

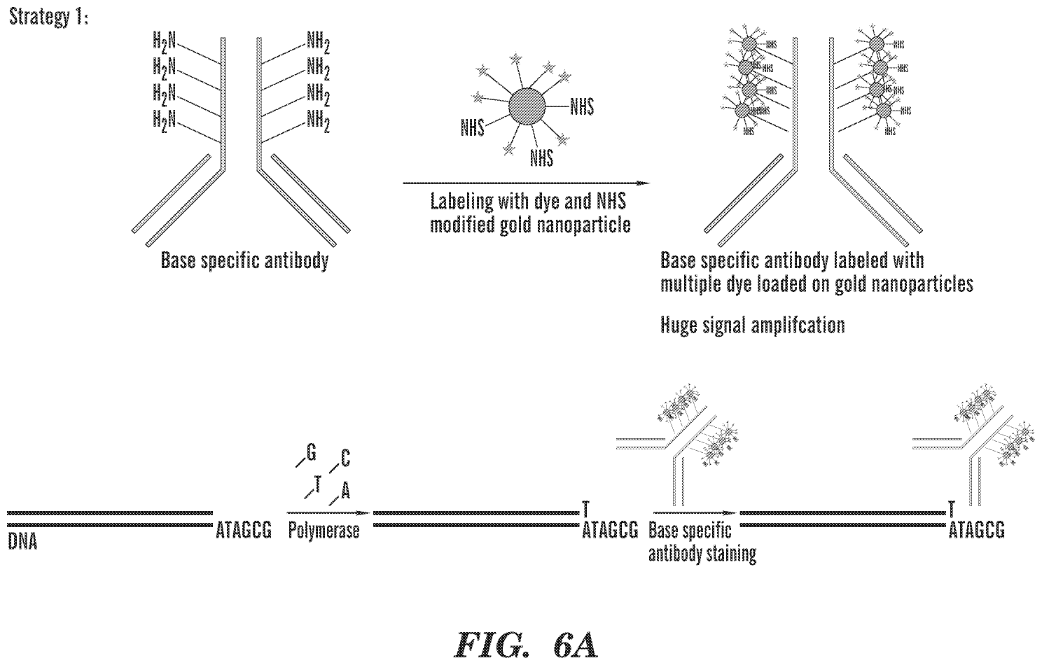

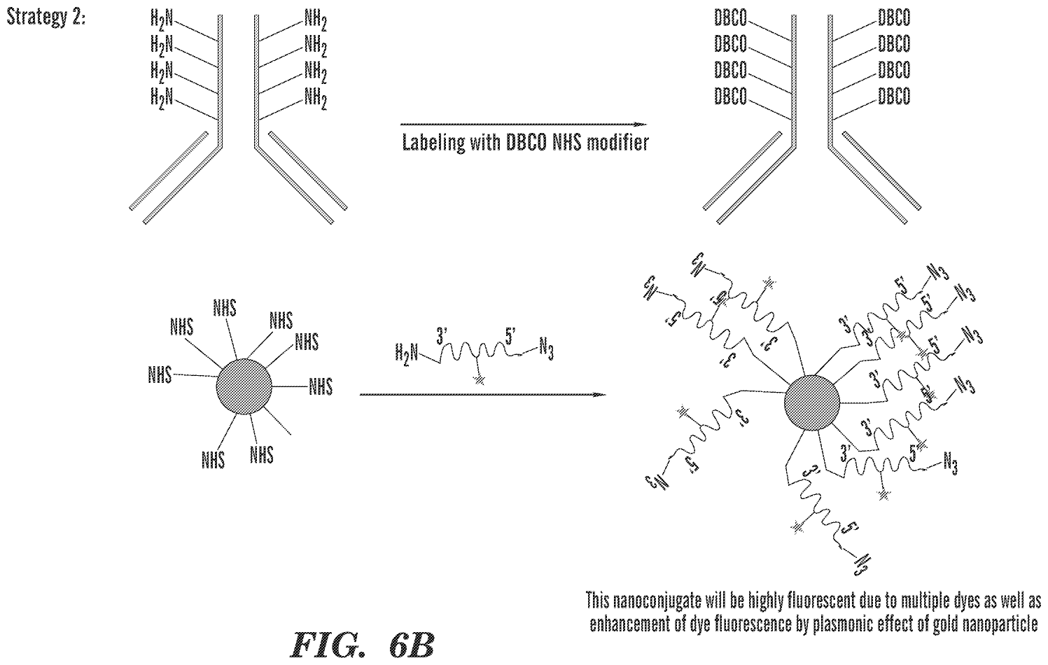

In many organisms, including humans, homologous chromosomes segregate into separate spatial locations in the nucleus, called Chromosome Territories (Cremer and Cremer. CSHL. 2010). FIG. 5A shows FISH probes targeting 6 genomic regions on each of Chr. 2, 3, and 5 in human fibroblasts (PGP1f). In each cell, the two homologs for each chromosome occupy separate territories. This separation allows chromoSEQ methods to phase as well as confirm phasing if the sequencing goes into regions where the maternal and paternal homologs differ in sequence. chromoSEQ can also determine if mutations are present on the same homologous chromosome. Sequencing of mutations on the same homolog would yield sequencing signal from proximal spots belonging to the same chromosome territory.

FIG. 5B shows Homolog specific Oligopaints (HOPs) can be used to identify parent of origin of homologous chromosomes. One set of HOPs targets the paternal single nucleotide polymorphisms (SNPs), here shown as C/G in magenta and the other set of HOPs targets the maternal SNPs, here shown as T/A in green. The HOPs will preferentially bind to one homolog. Images are of Chr19 HOPs in PGP1f cells. The paternal homolog stains in magenta and the maternal in green.

FIG. 5C is a schematic showing phasing with an exemplary embodiment. Here, primeSEQ is used but this method is also applicable to expoSEQ and mipSEQ for phasing. After primer hybridization (blue bar), in situ sequencing proceeds. The parent of origin information can be derived after the 2nd base is sequenced (G/A). HOPs can also be used to identify parent of origin in cases where sequencing does not yield sufficient information.

### Example 6: Results

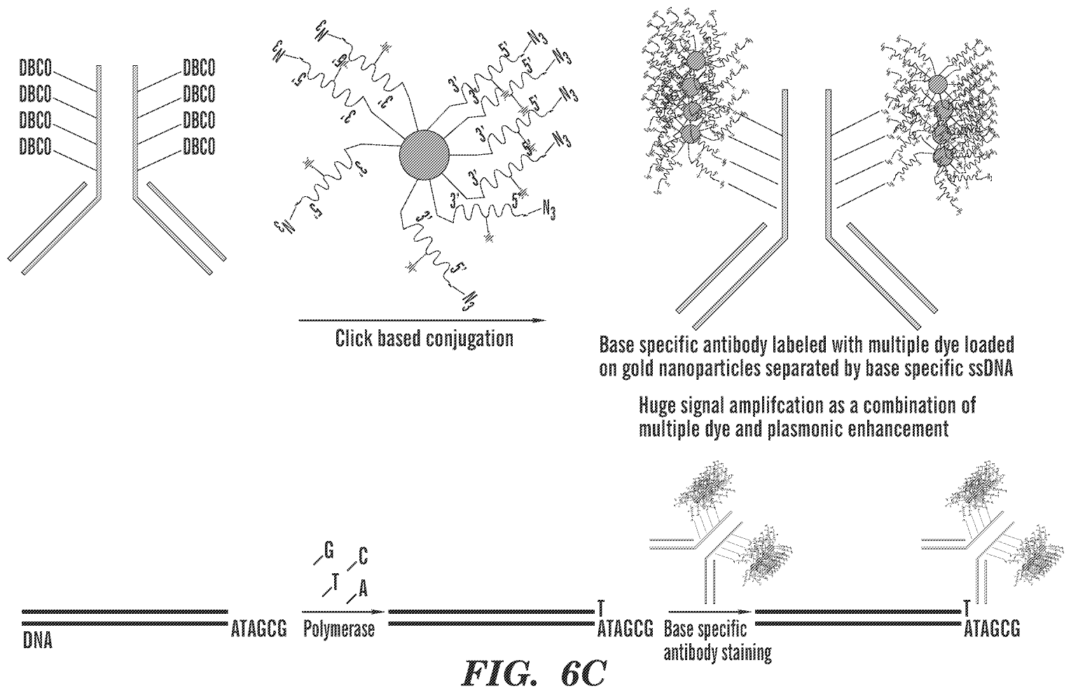

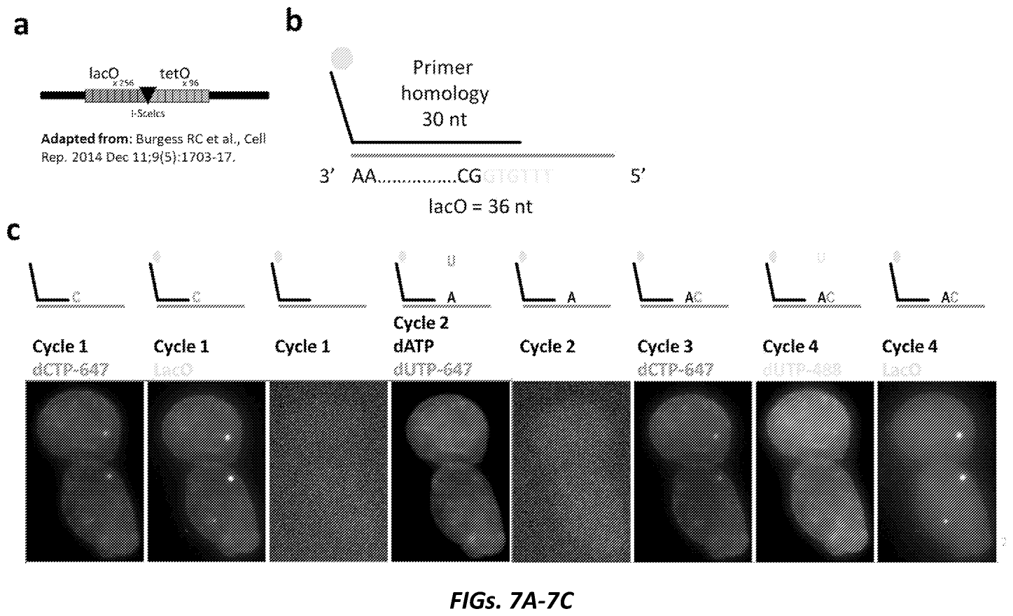

FIGS. 7A-7C show ChromoSEQ: ExpoSEQ 3 basepairs of lacO repeat in U2OS cells.

FIG. 7A Human U2OS cells containing an integrated construct with lacO and tetO sequence repeats were subjected to RASER-FISH (Brown, J. M. et al. Nat Commun 9, 3849 (2018)) protocol. RASER-FISH: Cells were grown on coverslips and allowed to adhere for 24 hours. Cell media was replaced with media containing bromo-deoxyuridine (BrdU) and bromo-deoxycytosine (BrdC) for 24-28 hours. Newly replicated DNA strands incorporate BrdU/BrdC during this time. Cells were then fixed with 4% formaldehyde in 1×PBS followed by irradiation under UV light (254 nm), causing DNA breaks on BrdU/BrdC incorporated strands. Exonuclease III was used to excise damaged/nicked DNA, resulting in single stranded genomic DNA.

FIG. 7B An oligonucleotide primer containing 30 nt of homology to 30/36 nt of lacO sequence was added to cells and hybridized overnight. The primer contains an overhang and a non-genome hybridizing sequence that can recruit fluorophore labeled oligos allowing for detection. Primer was designed to hybridize to 30/36 nt of lacO repeat such that single base extension off the primer results in the addition of 256 of the same nucleotide.

FIG. 7C chromoSEQ was then performed to sequence the first base (“Cycle 1”, expected to be Cytosine; (C)). Phusion DNA Polymerase was added with dCTP labeled with Alexa 647 and imaged, the panel shows 2 nuclei (Cycle 1 dCTP-647). A fluorophore labeled oligo (green) was then hybridized to flanking sequence to confirm location of lacO loci (Cycle 1 LacO). 647 nm signal was then bleached to extinguish signal before proceeding to sequencing next base (Cycle 1 Bleach). Next base (second) predicted to be added is Adenine. Phusion was added with unlabeled dATP and dUTP labeled with Alexa 647 and imaged, showing weak/non-existent signal in the same nuclei, as expected due to lack of fluorophore on Adenine. Note that lack of signal indicates that uridine was not added, as expected. Sample was then bleached. Next base (third) predicted to be added is Cytosine and signal appears after addition of Phusion and dCTP labeled with Alexa 647. Next base (fourth) predicted to be added is Adenine and signal is weak/non-existent after addition of Phusion and dUTP labeled with Alexa 488. Last panel shows signal from fluorophore labeled oligo used to detect the primer, demonstrating that primer is still present.

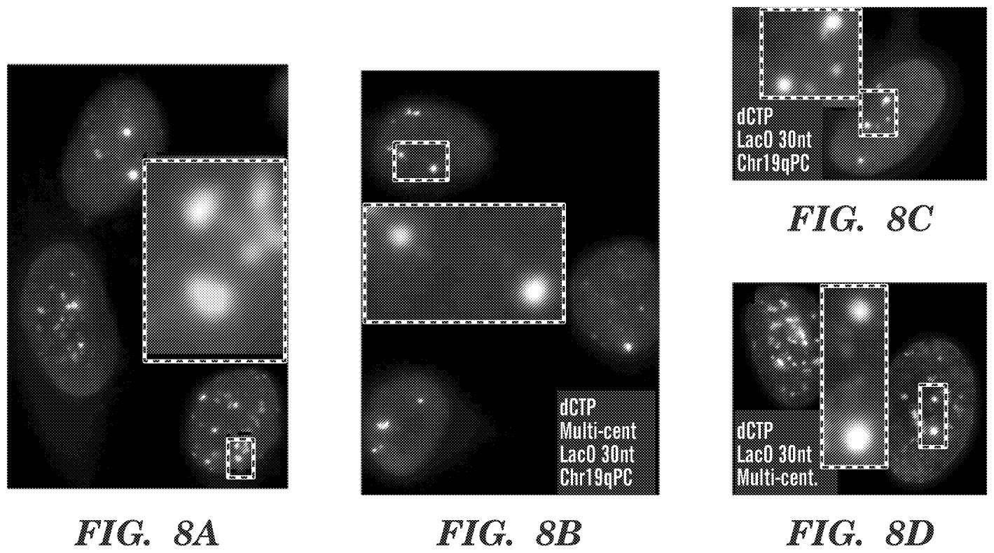

FIGS. 8A-8D ChromoSEQ; expoSEQ+primeSEQ preliminary data. U2OS cells containing LacO array (256 repeats) were subjected to RASER-FISH to yield single stranded genomic DNA to be used for direct sequencing off chromosomes.

FIG. 8A Multiple centromeric repeats (Multi-cent; magenta), 2.4 Mb pericentromeric region of Chr19 (yellow), and 36/36 nt of LacO repeat (green) were hybridized by oligos and visualized with labeled oligos targeting overhangs (streets). Image of 1st round of chromoSEQ using PfU High Fidelity DNA Polymerase with dCTP-Alexa647. Most LacO signal do not colocalize with magenta, indicating lack of sequencing from these targets. This was expected as the LacO 36nt oligo covers the entire LacO sequence.

FIG. 8B Similar experiment to FIG. 8A except using a shorter LacO oligo (30nt) replaces the 36nt. The next base added should be Cytosine. Here, LacO and magenta signal colocalize (white), indicating sequencing from these targets and addition of dCTP.

FIG. 8C Similar experiment to FIG. 8B except Multi-cent oligos were not added and labeled oligos were not added until after sequencing was performed.

FIG. 8D Similar experiment to FIG. 8C except Multi-cent oligos replaced Chr19qPC probes.

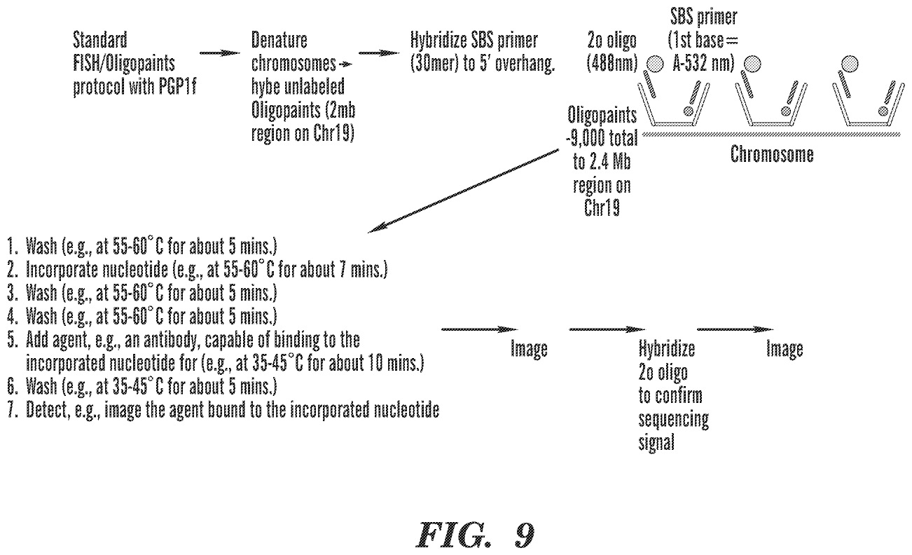

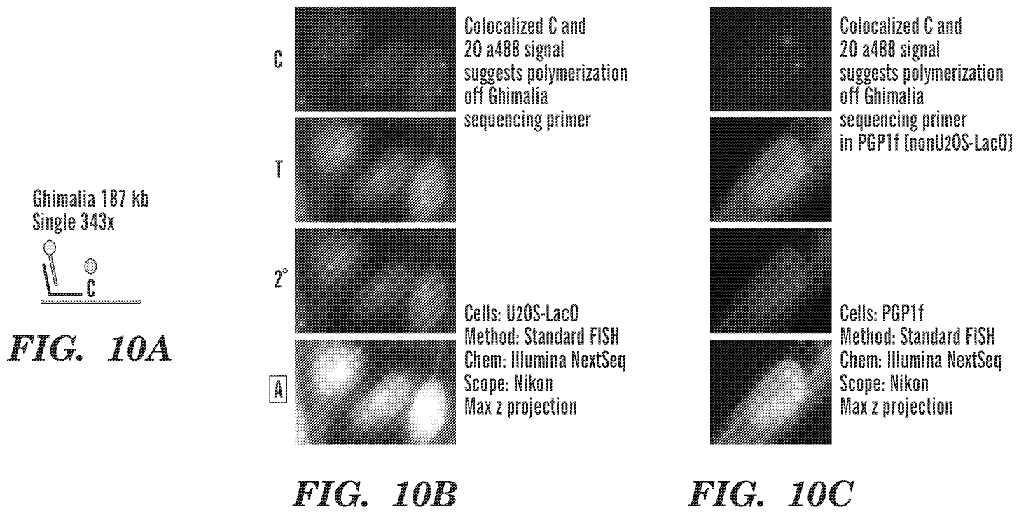

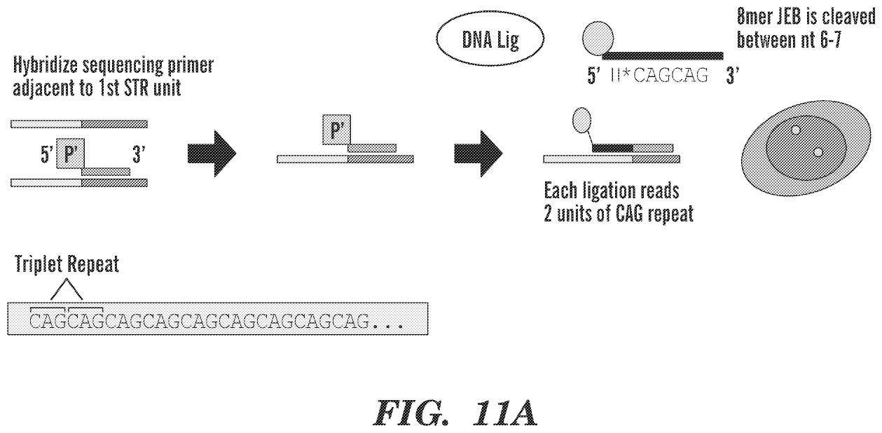

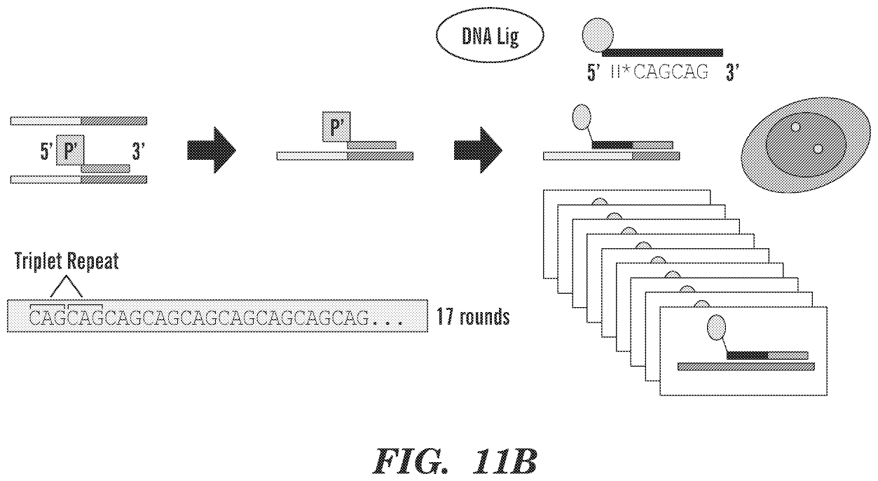

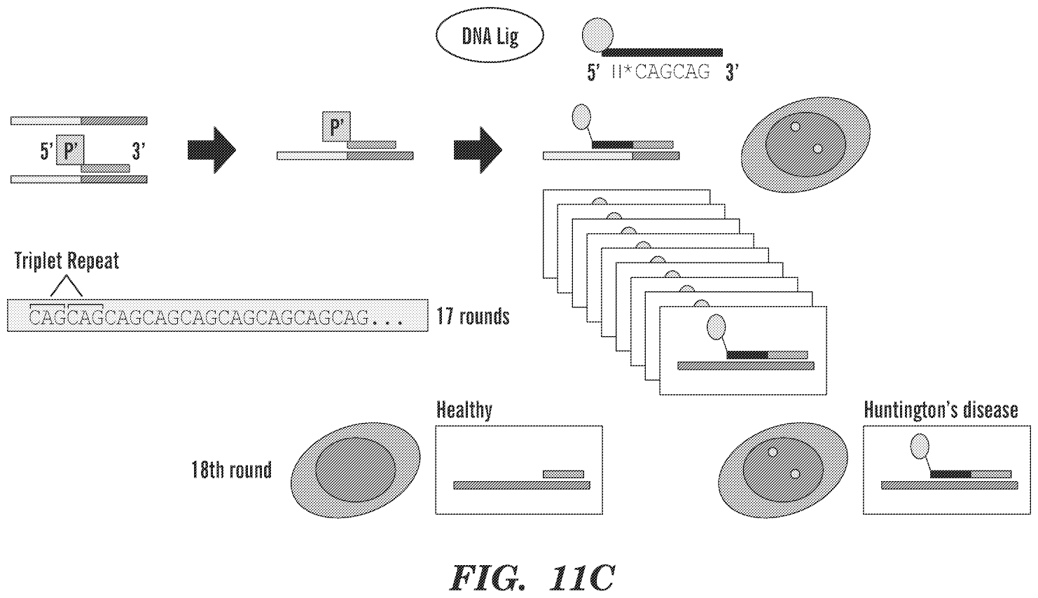

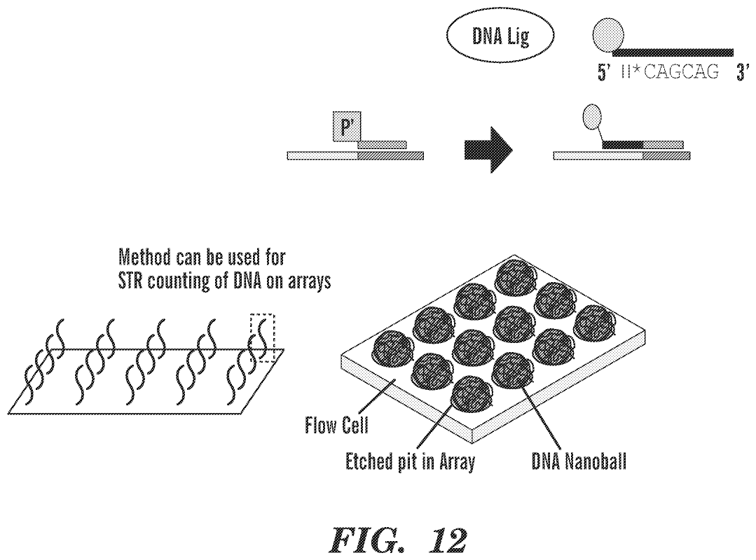

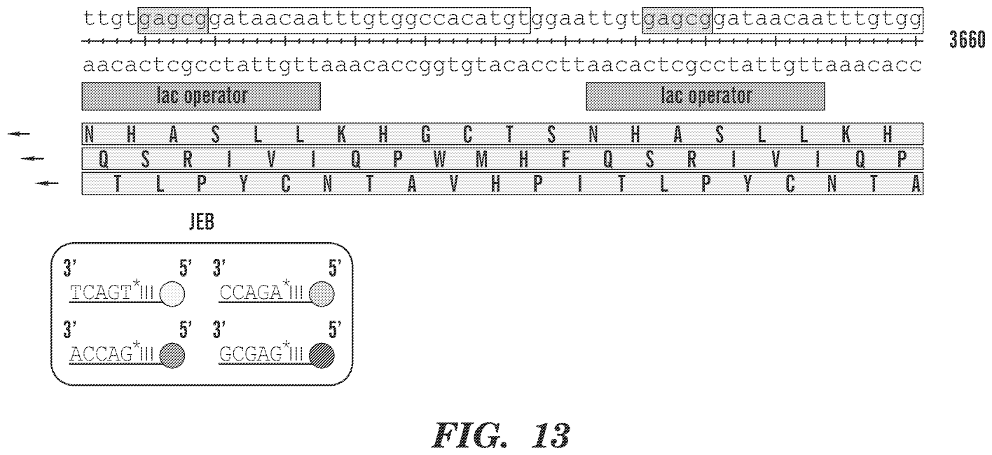

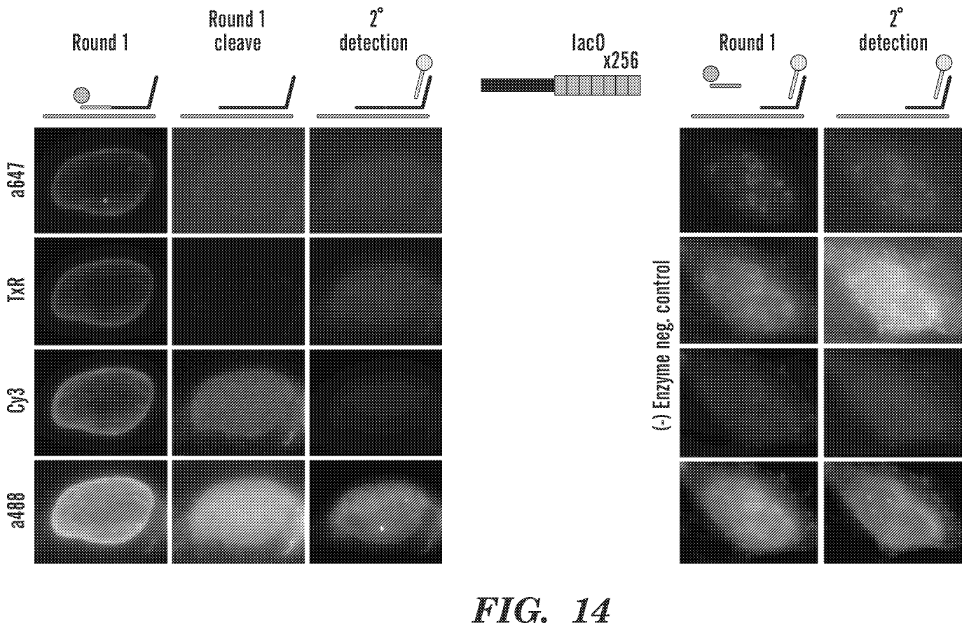

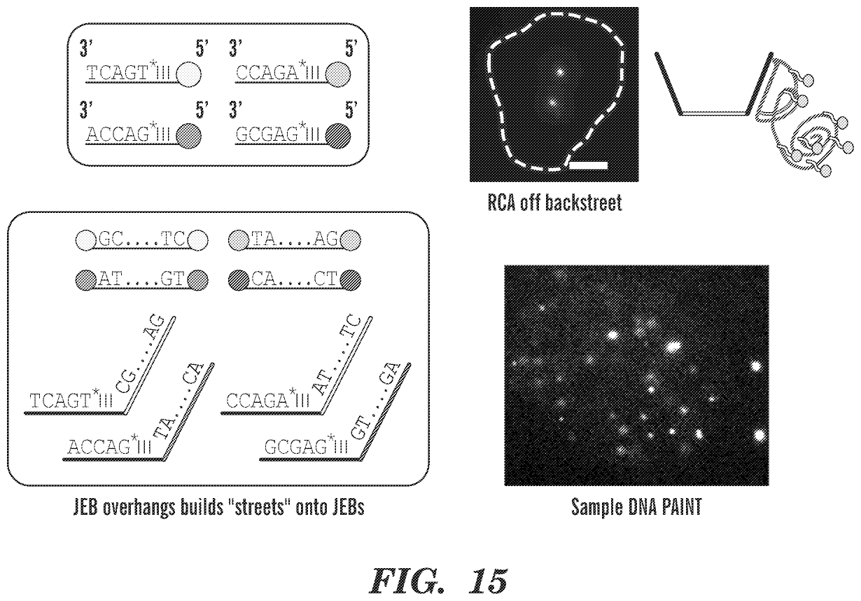

FIG. 9 is a schematic showing an exemplary method using sequencing-by-synthesis off Oligopaint streets (OligoFISSEQ synthesis based interrogation of targets). As shown in FIG. 9, the chromosome bound to Oligopaints and SBS primer is washed. The wash step could be done at a temperature that does not denature the Oligopaints and/or SBS primers. For example, the washing step could be at 55-60° C. for about 5-10 minutes. After the was step, a nucleotide is incorporated at the end of the SBS primer using any method known and available. The nucleotide can comprise a reversible terminator moiety. The step of incorporating the nucleotide can be at 55-60° C. for about 7-12 minutes. One or more washing steps are performed after incorporating the nucleotide. For example, a first washing step is to remove any residual reagents used for incorporating the nucleotide followed by a second wash step to optimize conditions for detecting the incorporated nucleotide. Each washing step can be at 55-60° C. for about 5-10 minutes. After the washing step(s), the agent, e.g., an antibody capable of binding the incorporated nucleotide and/or a moiety attached to the incorporated nucleotide is added. The agent comprises a detectable label, e.g., fluorophore. The reaction mixture is incubated for a sufficient period of time to allow the agent to bind to the nucleotide and/or the moiety attached to the nucleotide. Generally, the binding step is at 35-45° C. for about 10 minutes. After the binding step, a wash step is carried out to remove any excess inbound agent. This wash step is generally carried out at 35-45° C. for about 5 minutes. After washing, any bound agent is detected, e.g., by imaging a label conjugated to the agent. A secondary oligonucleotide is hybridized to the Oligopaints to confirm sequencing signal. The secondary oligonucleotide can comprise a detectable label and imaged.

All patents, patent applications, and publications identified are expressly incorporated herein by reference for the purpose of describing and disclosing, for example, the methodologies described in such publications that can be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of determining sequence information, and optionally positional information, on a chromosome in situ, the method comprising:
a) hybridizing a nucleic acid primer to a strand of a chromosome in situ under conditions that permit extension of the hybridized primer by a polymerase;
extending the hybridized primer in presence of a polymerase with a nucleotide to produce an extended hybridized primer, wherein the nucleotide is complementary to a nucleotide, directly downstream from the hybridized primer, on the chromosome strand hybridized to the primer, and wherein the nucleotide is conjugated with a moiety that permits detection of the nucleotide with an agent that specifically binds to the moiety and is capable of producing a detectable signal; and
contacting the moiety with the agent that specifically binds to the moiety and producing a detectable signal, thereby detecting incorporation of the nucleotide onto the hybridized primer;
or
b) providing a chromosome in situ that has a nicked strand, the nick leaving an extendable terminus;
extending the extendable terminus of the nicked strand of the chromosome in the presence of a polymerase with a nucleotide, wherein the nucleotide is complementary to a nucleotide, directly downstream from the extendable terminus, on a chromosome strand complementary to the nicked strand, and wherein the nucleotide is conjugated with a moiety that permits detection of the nucleotide with an agent that specifically binds to the moiety and is capable of producing a detectable signal; and
contacting the moiety with the agent that specifically binds to the moiety and producing a detectable signal, thereby detecting incorporation of the nucleotide onto the nicked strand.

2. The method of claim 1, wherein said moiety is an antigen or antibody, optionally the moiety is an antibody.

3. (canceled)

4. The method of claim 1, wherein the moiety is conjugated with the nucleotide via a cleavable linker and/or with one or more nanoparticles comprising a fluorophore.

5. (canceled)

6. The method of any one of the preceding claims, wherein the agent is conjugated with a detectable label, a docking nucleic acid strand, and/or a docking nucleic acid strand conjugated to a nanoparticle.

7. (canceled)

8. (canceled)

9. The method of claim 1, wherein the agent is conjugated with a docking nucleic acid strand conjugated to a nanoparticle and said detecting comprises:
(a) a step of producing an amplicon from the docking strand nucleic acid strand and detecting the amplicon;
(b) a step of producing a Signal amplification by Exchange Reaction (SABER) amplification from the docking strand nucleic acid strand and detecting the SABER amplified signal; or
(c) hybridizing a reporter nucleic acid strand with the docking nucleic acid strand, wherein the reporter nucleic acid strand comprises a detectable label.

10. (canceled)

11. (canceled)

12. The method of claim 1, wherein the agent is an antibody or nanobody.

13. The method of claim 1, wherein the method further comprises a step of extinguishing the detectable signal.

14. The method of claim 1, wherein the method further comprises:
(a) extending the extended hybridized primer in presence of a polymerase with a second nucleotide, wherein the second nucleotide is complementary to a nucleotide directly downstream from the extended hybridized primer on the chromosome strand to which the primer is hybridized, and wherein the nucleotide is conjugated with a second moiety that permits detection of the nucleotide with a second agent that specifically binds to the second moiety and is capable of producing a detectable signal; and
contacting the second moiety with the second agent that specifically binds to the moiety and producing a detectable signal, thereby detecting incorporation of the second nucleotide on to the extended hybridized primer;
or
(b) further extending the nicked strand in presence of a polymerase with a second nucleotide, wherein the second nucleotide is complementary to a nucleotide, directly downstream from the extended nicked strand, on the chromosome strand complementary to the nicked strand, and wherein the second nucleotide is conjugated with a second moiety that permits detection of the nucleotide with a second agent that specifically binds to the moiety and is capable of producing a detectable signal; and
contacting the second moiety with the second agent that specifically binds to the second moiety and producing a detectable signal, thereby detecting incorporation of the second nucleotide on to the nicked strand.

15. The method of claim 1, wherein the primer comprises a detectable label, a barcode sequence, a random mixture of nucleotides, a random sequence, at least one universal nucleobase, a homopolymer sequence, a quencher molecule, a moiety/label for isolating or purifying the primer.

16. (canceled)

17. (canceled)

18. The method of claim 1, wherein the primer hybridizes to a repetitive element, optionally the repetitive element is selected from the group consisting of a Long Interspersed Nuclear Element (LINE), Short Interspersed Nuclear Elements (SINE), SVA element, Alu element, centromeric repeat, trinucleotide repeat and a telomeric repeat.

19. (canceled)

20. (canceled)

21. The method of claim 1, wherein the chromosome is in a cell, optionally the cell is in a tissue or section thereof.

22. (canceled)

23. (canceled)

24. (canceled)

25. The method of claim 1, further comprising creating a nick on a strand of the chromosome prior to extending with the polymerase and the nucleotide.

26. The method of claim 1, further comprising, prior to extending with the polymerase and the nucleotide, contacting the nicked strand with an exonuclease.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. A method of determining sequence information, and optionally spatial information, on a chromosome in situ, the method comprising:
a) hybridizing a molecular inversion probe (MIP) to a strand of a chromosome in situ under conditions that permit extension of the MIP, wherein a first end of the MIP hybridizes to a first region of the chromosome strand and the second end of the MIP hybridizes to a second region of the same chromosome strand and wherein the first and the second regions are separated by at least one nucleotide;
extending one end of the hybridized MIP by at least one nucleotide in presence of a polymerase with a nucleotide, wherein the nucleotide is complementary to a nucleotide, directly downstream from the first or second end of the hybridized MIP, on the chromosome strand hybridized to the MIP;
ligating together the two ends of the hybridized, extended MIP;
amplifying the ligated MIP to generate a template strand; and
sequencing the template strand
or
b) hybridizing a nucleic acid probe to a strand of a chromosome in situ, wherein the nucleic acid probe comprises a barcode sequence, a docking sequence, and a sequence complementary to a nucleotide sequence of the chromosome strand;
hybridizing a molecular inversion probe (MIP) to the docking sequence of the probe under conditions that permit extension of the MIP, wherein a first end of the MIP hybridizes to a first region of the probe and the second end of the MIP hybridizes to a second region of the probe and wherein the first and the second regions are separated by at least one nucleotide;
extending one end of the hybridized MIP by at least one nucleotide in presence of a polymerase with a nucleotide, wherein the nucleotide is complementary to a nucleotide, directly downstream from the first or second end of the hybridized MIP, on the probe;
ligating together two ends of the hybridized, extended MIP;
amplifying the ligated MIP to generate a template strand; and
sequencing the template strand.

45. The method of claim 44, wherein said amplifying the MIP comprises rolling circle amplification or SABER amplification.

46. (canceled)

47. The method of claim 44, wherein the MIP comprises a barcode sequence and/or a priming sequence.

48. (canceled)

49. The method of claim 44, wherein said sequencing the template strand is by a fluorescence-based sequencing method, sequencing by ligation, sequencing by hybridization, and/or sequencing by synthesis.

50. (canceled)

51. (canceled)

52. (canceled)

53. The method of claim 44, wherein the MIP hybridizes to a repetitive element, optionally the repetitive element is selected from the group consisting of a Long Interspersed Nuclear Elements (LINE), Short Interspersed Nuclear Elements (SINE), SVA element, Alu element, centromeric repeat, trinucleotide repeat and a telomeric repeat.

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. The method of claim 1, wherein the chromosome is in a cell, optionally the cell is in a tissue or section thereof.

70. (canceled)

71. (canceled)

72. (canceled)

73. (canceled)

74. (canceled)

75. (canceled)

76. (canceled)

77. (canceled)

78. A method for counting the short tandem repeats (STRs) in a target nucleic acid, the method comprising: (i) hybridizing a first oligonucleotide directly adjacent to a STR region in a target nucleic acid; (ii) ligating a second oligonucleotide to the first oligonucleotide that is hybridized with the target, wherein the second oligonucleotide comprises a nucleotide sequence complementary to one STR unit and wherein the second oligonucleotide comprises a detectable label; (iii) detecting the detectable label; (iv) cleaving the ligated second oligonucleotide, e.g., with a nicking enzyme to release the detectable label from the ligated oligonucleotide; and (v) repeating steps (ii)-(iv), until a detectable label is not detected in step (iv).

79. (canceled)

80. (canceled)
